(19) United States
(12) Patent Application Publication
(10) Pub. No.: US 2004/0175381 A1
(43) Pub. Date: Sep. 9, 2004

(54) Inducing tolerance in primates

(75) Inventors: Dawn Winsor-Hines, Framingham, MA (US); Patricia Rao, Acton, MA (US); Douglas J. Ringler, Cambridge, MA (US)

Correspondence Address: LAHIVE & COCKFIELD, LLP., 28 STATE STREET, BOSTON, MA, 02109, US

(73) Assignee: TolerRx, Inc., Cambridge, MA

(21) Appl. No.: 10/731,984

(22) Filed: Dec. 9, 2003

Related U.S. Application Data
(60) Provisional application No. 60/431,839, filed on Dec. 9, 2002.

(51) Int. Cl. A61K 39/395

(52) U.S. Cl. 424/144.1

(57) ABSTRACT

The present invention is based, at least in part, on the finding that tolerance can be induced by inhibition of CD4+ and CD8+ T cells. Accordingly, the methods of the invention are useful in treating a primate, e.g., a human, by inhibiting CD4+ and CD8+ T cells to induce tolerance to at least one antigen, e.g., self or foreign, such as for inducting tolerance in a primate against a transplanted antigen, e.g., an allogeneic or xenogeneic transplanted antigen.

### RELATED APPLICATIONS

This application claims the benefit of U.S. Provisional Application, 60/431,839, filed Dec. 9, 2002, titled “Introducing Tolerance to Proteins in Primates,” the entire contents of which is incorporated herein by reference.

### BACKGROUND OF THE INVENTION

This invention relates to tolerance induction and more particularly to inducing tolerance in a primate against an antigen(s) and in particular a foreign antigen.

There have been numerous attempts to induce tolerance against foreign and self antigens in a primate. For example, in the field of transplantation, there is a need to induce tolerance to foreign antigens in a transplant so as to prevent its rejection At present rejection can only be prevented by the use of long-term (chronic) immunosuppression which carries risks of infection, cancer and drug toxicity.

In addition, in the treatment of a patient with a therapeutic protein, in many cases, treatment becomes less effective or totally ineffective as a result of an immune response to that foreign protein. As a result, there is a need for inducing tolerance in a primate against such protein in order to enable a more effective use of the therapeutic protein.

Thus, there is a need for a treatment that induces tolerance in a primate against an antigen(s) and in particular a foreign protein(s) in the above cases as well as other cases.

### SUMMARY OF THE INVENTION

In accordance with an aspect of the present invention, there is provided a process for tolerizing a primate against an antigen(s) by use of a CD4 antibody or fragment thereof.

In one aspect, the invention pertains to process for treating a primate to induce tolerance to at least one antigen by administering to the primate at least one anti-CD4 antibody or CD4 binding fragment thereof and at least one compound that inhibits CD8+ T cells each in an amount and for a time effective to induce tolerance against at least one antigen, the anti-CD4 antibody or fragment being present in said primate when the antigen is present in the primate and the anti-CD4 antibody being administered in an initial dose of at least 40 mg/kg, such that tolerance against said antigen is induced.

In one embodiment, the antigen is a foreign antigen.

In another aspect, the invention pertains to a process for inducing tolerance in a primate to a transplanted antigen byadministering to a primate at least one anti-CD4 antibody or CD4 binding fragment thereof and at least one compound that inhibits CD8+ T cells each in an amount and for a time effective to induce tolerance against the transplant, the anti-CD4 antibody or fragment being present in said primate when the transplanted antigen is present in the primate and the anti-CD4 antibody being administered in an initial dose of at least 40 mg/kg, such that tolerance to the transplanted antigen is induced

### BRIEF DESCRIPTION OF THE DRAWINGS

FIG. 1A shows the amino acid sequence of the first embodiment of TRX1 antibody light chain. FIG. 1B shows the nucleotide sequence of the first embodiment of TRX1 antibody light chain. FIG. 1C shows the amino acid sequence of the first embodiment of TRX1 antibody light chain with and without a leader sequence. FIG. 1D shows the amino acid sequence of the first embodiment of TRX1 antibody heavy chain. FIG. 1E shows the nucleotide sequence of the first embodiment of TRX1 antibody heavy chain. FIG. 1F shows the amino acid sequence of the first embodiment of TRX1 antibody heavy chain with and without a leader sequence.

FIG. 2A shows the amino acid sequence of another embodiment of TRX1 antibody light chain. FIG. 2B shows the nucleotide sequence of another embodiment of TRX1 antibody light chain. FIG. 2C shows the amino acid sequence of another embodiment of TRX1 antibody light chain with and without a leader sequence. FIG. 2D shows the amino acid sequence of another embodiment of TRX1 antibody heavy chain. FIG. 2E shows the nucleotide sequence of another embodiment of TRX1 antibody heavy chain. FIG. 2F shows the amino acid sequence of another embodiment of TRX1 antibody heavy chain with and without a leader sequence.

FIG. 3A shows the amino acid sequence of another embodiment of TRX1 antibody light chain. FIG. 3B shows the nucleotide sequence of another embodiment of TRX1 antibody light chain. FIG. 3C shows the amino acid sequence of another embodiment of TRX1 antibody light chain with and without a leader sequence. FIG. 3D shows the amino acid sequence of another embodiment of TRX1 antibody heavy chain.

FIG. 3E shows the nucleotide sequence of another embodiment of TRX1 antibody heavy chain. FIG. 3F shows the amino acid sequence of another embodiment of TRX1 antibody heavy chain with and without a leader sequence.

FIG. 4A shows the amino acid sequence of another embodiment of TRX1 antibody light chain. FIG. 4B shows the nucleotide sequence of another embodiment of TRX1 antibody light chain.

FIG. 4C shows the amino acid sequence of another embodiment of TRX1 antibody light chain with and without a leader sequence. FIG. 4D shows the amino acid sequence of another embodiment of TRX1 antibody heavy chain. FIG. 4E shows the nucleotide sequence of another embodiment of TRX1 antibody heavy chain. FIG. 4F shows the amino acid sequence of another embodiment of TRX1 antibody heavy chain with and without a leader sequence.

FIG. 5 shows the sequence of the heavy chains of the humanized CD8 antibody used in Example 5.

FIG. 6 shows the sequence of the light chains of the humanized CD8 antibody used in Example 5.

### DETAILED DESCRIPTION OF THE INVENTION

The antigen(s) as to which tolerance is induced may be a self antigen or a foreign antigen and in particular a foreign antigen(s).

As used herein, the term “tolerize” or “tolerant” or “tolerance” includes refractivity to activating receptor-mediated stimulation. Such refractivity is generally antigen-specific and persists after exposure to the tolerizing antigen has ceased. For example, tolerance is characterized by lack of cytokine production, e.g., IL-2 upon subsequent exposure to the tolerizing antigen. Tolerance can occur to self antigens or to foreign antigens. In one embodiment, the a tolerant primate does not produce an adverse immune response to the antigen over a period of time after treatment with a tolerizing agent is stopped even when subsequently challenged with the antigen and/or when the antigen remains present in the primate, but is capable of providing an immune response against other antigens. In one embodiment, tolerance is induced in the absence of a therapeutic level of a general immunosuppressant.

For example, the foreign antigen may be one or more of the following types of antigens:

(i) a foreign antigen(s) present on transplanted tissue or cells, including tissue or cells present in an organ wherein the transplant may be allogeneic or xenogeneic;

(ii) a therapeutic agent (which also includes therapeutic agents used for disease prevention) that produces an immune response in a primate, which immune response diminishes the ability of the agent to function as a therapeutic agent. Such agents include, but are not limited to, delivery vehicles, such as vectors used in gene therapy; active agents such as proteins delivered to the primate (e.g., recombinant proteins such as monoclonal antibodies, enzymes, clotting factors) and some small molecule drugs or proteins produced from an agent delivered to the primate, such as in gene therapy.

The foreign antigens against which tolerance is induced in accordance with the present invention are not foreign antigens as present in disease-causing bacteria, fungi, viruses, etc. that infect a host, i.e., the term foreign antigen does not include a foreign antigen as part of an organism that infects a primate and causes a disease or disorder.

The CD4 antibody or CD8 antibody in the case where a CD8 antibody is used, is preferably a monoclonal antibody (or fragment thereof that retains the ability to bind to CD4 or CD8, respectively). The antibody may be a human antibody or a non-human antibody, with non-human antibodies including humanized antibodies, chimeric antibodies, murine antibodies, etc.

The CD4 antibody or appropriate fragment thereof is administered to a primate in an amount and for a time effective to induce tolerance against a foreign or self antigen and preferably a foreign antigen. Anti-primate CD4 antibodies are known in the art as are methods of making such antibodies.

In one embodiment, the anti-CD4 antibody is administered prior to exposure (or systemic exposure) to the antigen to which tolerance is desired. In another embodiment, the anti-CD4 antibody is administered simultaneously with the antigen to which tolerance is desired.

The compound that inhibits CD8+ T cells inhibits the activity of CD8+ T cells, e.g., by reducing their number or by inhibiting their effector function. In one embodiment, a compound that inhibits CD8+ T cells specifically inhibits CD8+ T cells. In another embodiment, a compound that inhibits CD8+ T cells does not significantly inhibit or deplete Treg cells. Such a compound may be an antibody that does or does not deplete CD8+ T cells. Anti-primate CD8 antibodies are known in the art as are methods for making such antibodies. The compound that inhibits CD8+ T-cells may be a compound (other than an antibody) that inhibits such CD8+ T cells (such compound other than an antibody may or may not deplete CD8+ T cells. Exemplary non-antibody compounds include, e.g., beta-galactoside-binding protein (Blaser et al. 1998. Eur J Immunol. 28:2311).

In one embodiment, the compound that inhibits CD8+ T cells is administered prior to administration of the anti-CD4 antibody. In another embodiment, the compound that inhibits CD8+ T cells is administered simultaneously with the anti-CD4 antibody. In another embodiment, the compound that inhibits CD8+ T cells is subsequent to administration of the anti-CD4 antibody.

As used herein, the term “regulatory T cell” includes T cells which produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at levels less than or equal to that produced by Th1 or Th2 cells, e.g., an order of magnitude less than in Th1 or Th2 cells. Regulatory T cells can be found in the CD4+CD25+ population of cells (see, e.g., Waldmann and Cobbold. 2001. Immunity. 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody, plus anti-CD28 antibody).

As representative examples of compounds (other than antibodies) that inhibit CD8+ T cells there may be mentioned: Rapamycin (sirolimus); CellCept® (mycophenolate mofetil). A compound such as cyclosporin is preferably not used in that although it inhibits CD8+ T cells, such compound also has a substantial effect with respect to depletion of Treg cells.

The present invention has particular applicability to inducing tolerance in a primate with respect to a transplant and preferably such primate is a human. The transplant may be allogeneic or xenogeneic.

In accordance with a preferred embodiment, each of the CD4 antibody or appropriate fragment thereof and the CD8 inhibiting compound is administered over a period of time in order to maintain in the primate appropriate levels of such antibody or fragment and compound over a period of time that is sufficient to induce tolerance.

In general, the CD4 antibody (or fragment thereof) is administered in an initial dose of at least about 40 mg/kg, preferably at least about 50 mg/kg and more preferably in an amount of at least about 70 mg/kg.

In one preferred embodiment, the initial dose is at least 400 mg/kg, preferably at least about 500 mg/kg and in a particular embodiment in an amount of at least about 700 mg/kg.

The initial dose of the CD4 antibody may be administered in one or more doses over a twenty-four hour period and preferably in one dose over twenty-four hours.

As used herein in reference to a dosage amount, a dose is the total amount of the CD4 antibody administered over a twenty-four hour period, even if administered more than once in 24 hours.

In most cases, after the initial dose, the CD4 antibody (or appropriate fragment thereof) is administered in one or more follow-up doses over several day(s), with each follow-up dose being administered in one or more doses in a twenty-four hour period. The follow-up dose(s) is generally provided in an amount to return serum levels of the CD4 antibody to those that were achieved by the initial dose.

In a preferred embodiment, the minimum follow-up dose or doses of the CD4 antibody is in an amount that is at least equal to the amounts hereinabove described and may or may not be identical to the dose given as the original or initial dose. Thus, a follow-up dose of the CD4 antibody is generally at least 40 mg/kg, preferably at least 50 mg/kg, and more preferably at least 70 mg/kg. As hereinabove described, in one preferred embodiment, the follow-up dose(s) is at least 400 mg/kg, preferably at least 500 mg/kg, and in a particular embodiment at least 700 mg/kg.

If there is more than one follow-up dose of the CD4 antibody, each such additional follow-up dose over a 24-hour period may be the same or different than another follow-up dose.

The number of follow-up doses of the CD4 antibody will vary, but in a preferred embodiment, there is generally at least one follow-up dose and in most cases there is no more than seven follow-up doses, i.e., the total number of doses generally does not exceed eight daily doses.

The total period over which the CD4 antibody is administered generally does not exceed four weeks and more preferably does not exceed three weeks. In many cases, tolerance can be achieved by using an initial dose and one or more follow-up doses over a period that does not exceed two weeks.

Although, in accordance with the present invention, initial tolerance to an antigen(s) can be achieved in a primate in a period of no more than four weeks, in some cases, periodic follow-up treatments with the CD4 antibody may be administered in order to maintain tolerance.

As hereinabove described, at least one CD4 antibody (or appropriate fragment thereof) is delivered in an amount that is at least sufficient to induce tolerance in a primate against an antigen(s) and in a preferred embodiment against a foreign antigen. The maximum amount is of course limited by safety considerations. In general, the daily dosage of CD4 antibody would be less than 6000 mg.

The number of follow-up doses and the spacing thereof will be determined, in part, by the half life of the at least one CD4 antibody. Although the present invention is not to be limited thereby, it is believed that the CD4 antibody should be initially delivered in an amount to achieve antibody serum levels that exceed the amount required to saturate all of the CD4 of the primate being treated, with follow-up doses being given at times to maintain such excess over a period that induces tolerance in the primate against the foreign antigen(s).

In a preferred embodiment, the CD4 antibody is a CD4 antibody that would have a reduced effector (i.e. lytic) function as compared to human IgG1. As representative examples of antibodies that would have reduced effector function, there may be mentioned antibodies that have an Fc portion that is aglycosylated and/or that has reduced binding to the Fc receptor and/or is non-lytic.

In one embodiment, a CD4 antibody with a reduced effector function is a non-depleting CD4 antibody. As used herein, “a non-depleting CD4 antibody” is a CD4 antibody that depletes less than 50% of CD4 cells and preferably less than 10% of CD4 cells.

The CD8 inhibiting compound is administered to the primate during the initial treatment with the CD4 antibody in an amount effective to reduce the action and/or level of CD8+ T cells in the primate. Such amounts may be lower than the amounts used for the CD4 antibody. The CD8 inhibiting compound may be used at the same time as the CD4 antibody or may be used at different times. The CD8 inhibiting compound may be administered on different days or on the same day as the CD4 antibody. As hereinabove described, the CD8 inhibiting compound may be an antibody (or fragment thereof) or a compound other than an antibody. The treatment with the CD8 inhibiting compound is performed during the initial treatment (including initial follow-up doses); however, if further treatment with CD4 antibody is used after the initial treatment period (including follow-up doses), such further treatment may be performed with or without treatment with the CD8 inhibiting compound.

In treating a primate and in particular a human, each of the CD4 antibody and the CD8 inhibiting compound alone or in combination with each other may be employed in combination with a pharmaceutically acceptable carrier. A composition that contains a CD4 antibody and/or CD8 inhibiting compound may include other ingredients, for example, stabilizers and/or other active agents.

The use of a CD4 antibody and a CD8 inhibiting compound to induce tolerance against an antigen(s) in a primate in accordance with the present invention provides tolerance against one or more antigens and the primate is capable of immunologically responding to other antigens. Thus, in this respect, the primate is made tolerant to one or more antigens, and the immune system is capable of providing an immune response against other foreign antigens whereby the primate is not immunocompromised.

In the preferred embodiment where tolerance is induced against a foreign antigen, each of the CD4 antibody and the CD8 inhibiting compound, alone or in combination with each other is administered to the primate prior to, in conjunction with or subsequent to the foreign antigen being delivered to the primate. In a preferred embodiment, the primate is provided with the CD4 antibody and the CD8 inhibiting compound at a time such that both are present in the primate when the antigen(s) against which tolerance is to be induced is also present in the primate. In a particularly preferred embodiment, each of the CD4 antibody (or fragment thereof) and the CD8 inhibiting compound is delivered to the primate prior to the primate coming into contact with the foreign antigen(s) to which the primate is to be tolerized or within a few hours or less than one day thereafter. In a preferred embodiment, each of the CD4 antibody and the CD8 inhibiting compound is administered to the primate no more than about two, preferably no more than one day prior to the primate receiving the foreign antigen.

As hereinabove indicated, in one embodiment, a primate is tolerized against a therapeutic protein that is to be used in treating the primate. Such therapeutic protein may be a therapeutic antibody (other than the CD4 antibody), which therapeutic antibody may be a human antibody, humanized antibody, chimeric antibody or a non-human antibody; an enzyme such as one used for replacement therapy; a hormone; clotting factor; a protein produced in gene therapy; a gene therapy delivery vehicle such as a vector used in gene therapy (for example, an adenovirus vector).

The foreign antigen(s) may be present in a transplanted organ, or in transplanted cells used in cell therapy, or in other tissue transplants, such as skin.

The treatment of a primate, in particular, a human, in order to tolerize the primate against a foreign antigen by use of a CD4 antibody and a CD8 inhibiting compound may be accomplished in some cases without adjunct therapy, such as a bone marrow transplant to promote acceptance of a foreign antigen and/or immunosuppression.

In some cases, adjunct therapy may also be employed. For example, as part of a transplant procedure, immunosuppression with an appropriate immunosuppressant may be used but by employing the present invention, chronic immunosuppression is not required. In addition, if used after or during the tolerizing procedure, in some cases, the immunosuppressant may be used with less than the amount required to provide for effective immunosuppression.

In one non-limiting embodiment, the CD4 antibody is preferably a TRX1 antibody or one that binds to the same epitope as TRX1, and such CD4 antibody is preferably used with the dosing regimen as hereinabove described.

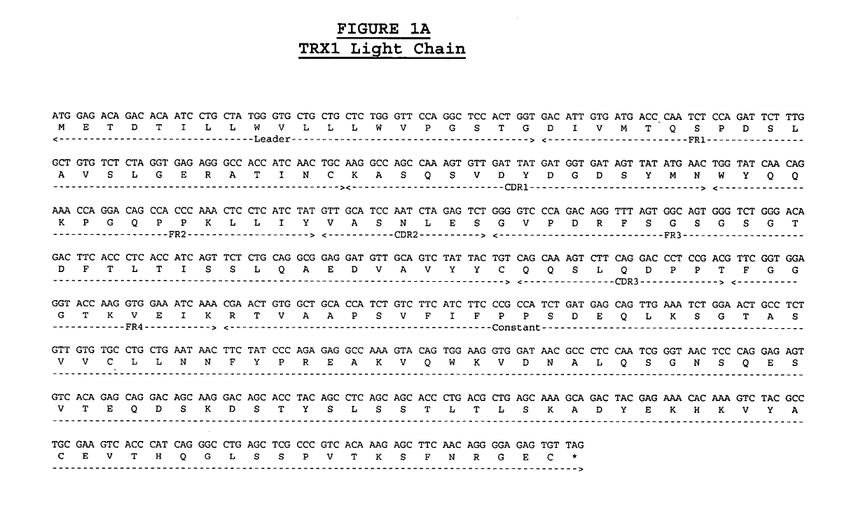

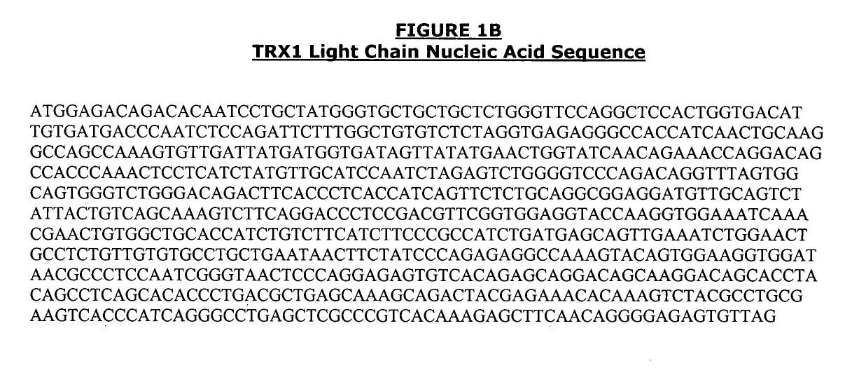

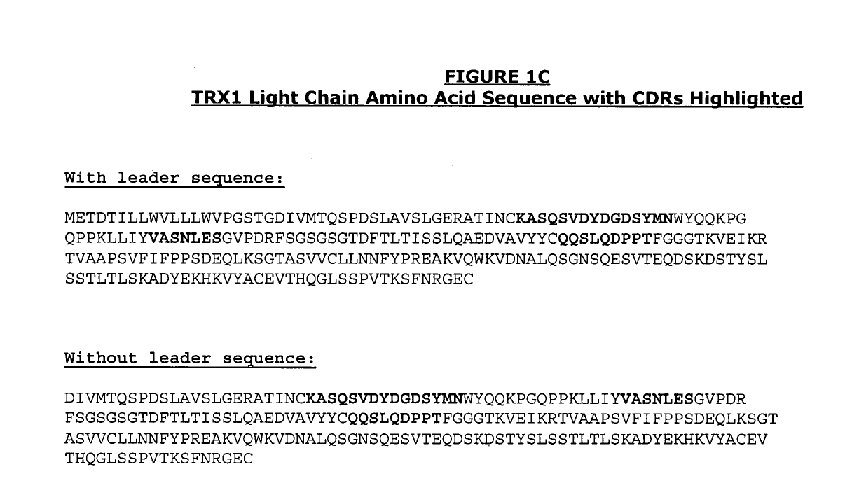

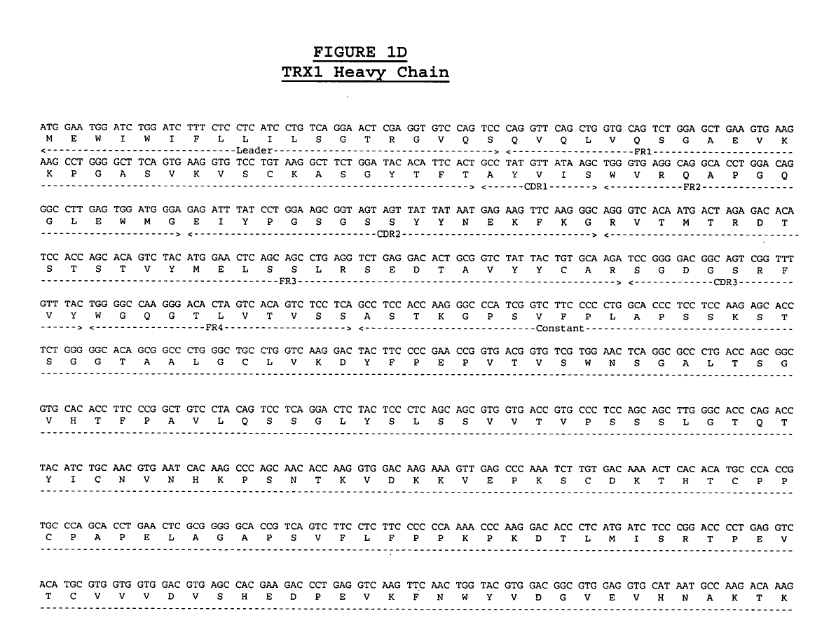

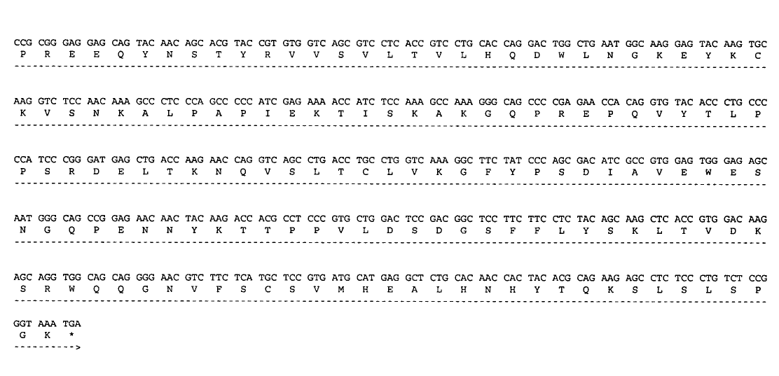

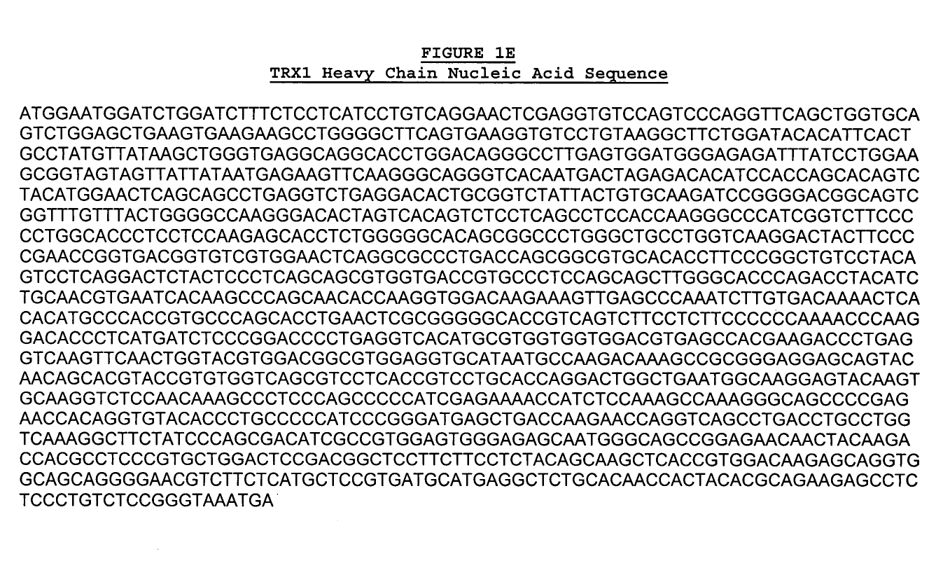

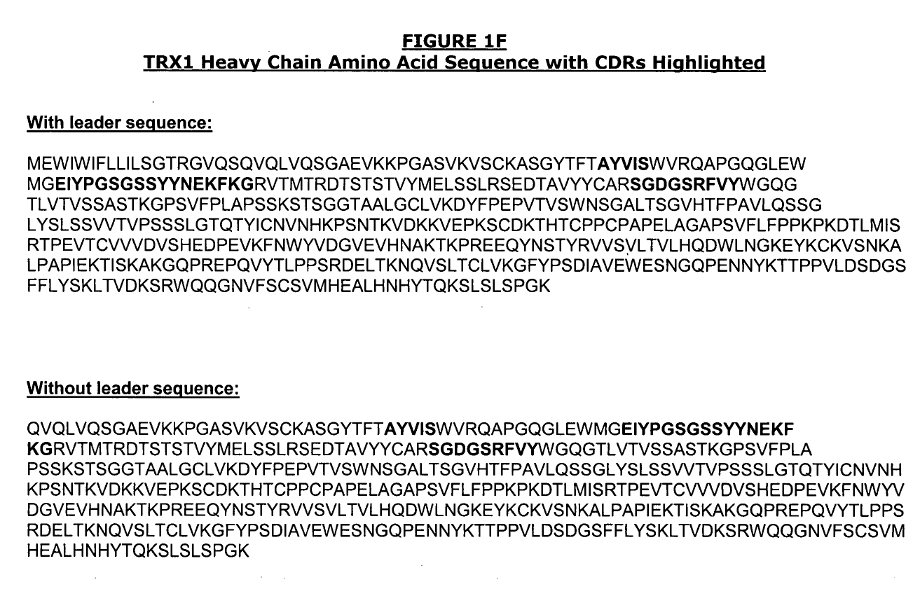

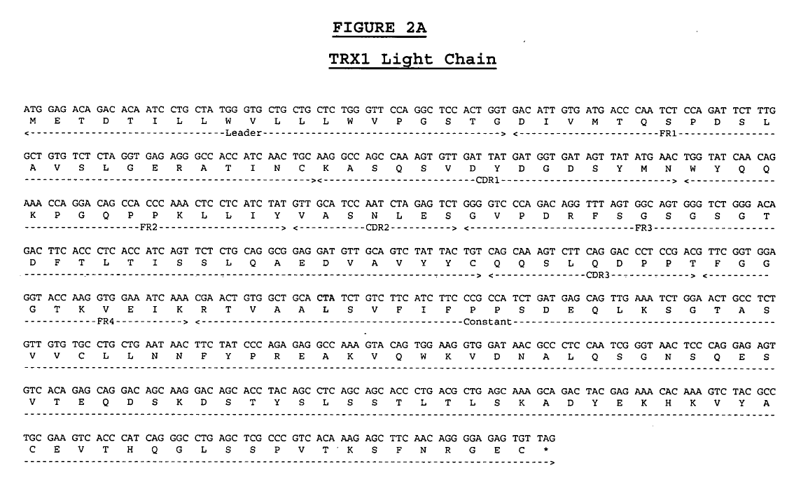

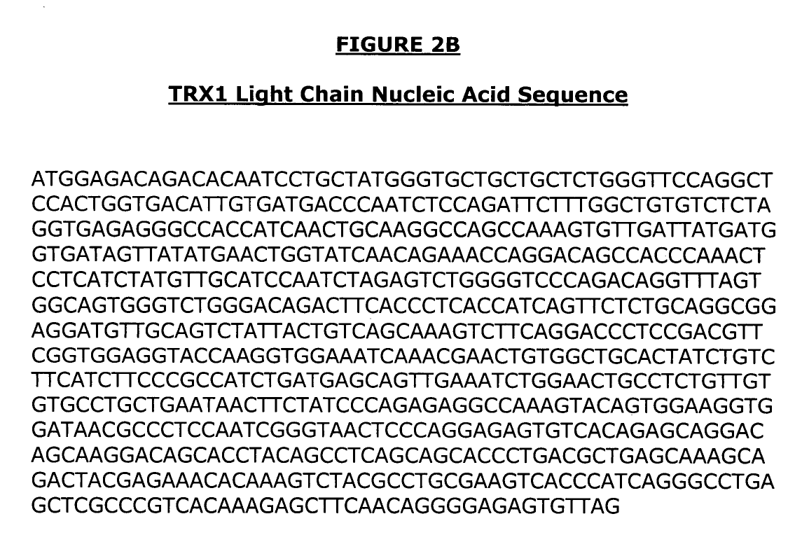

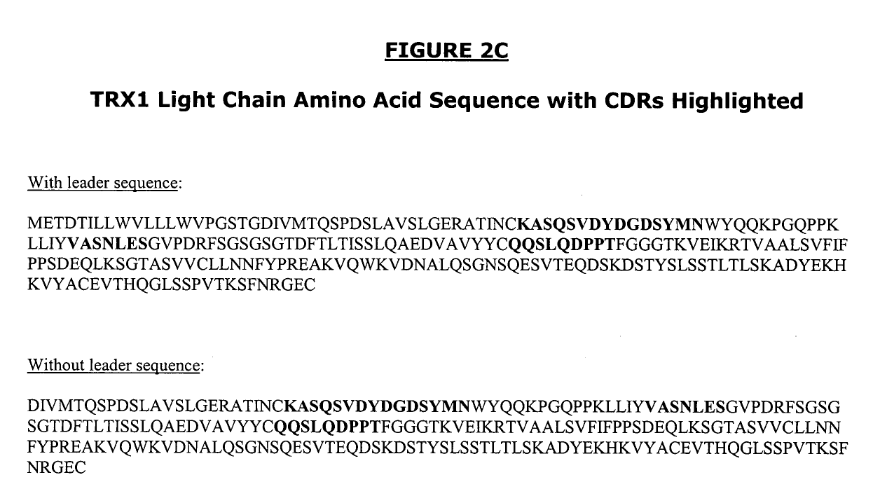

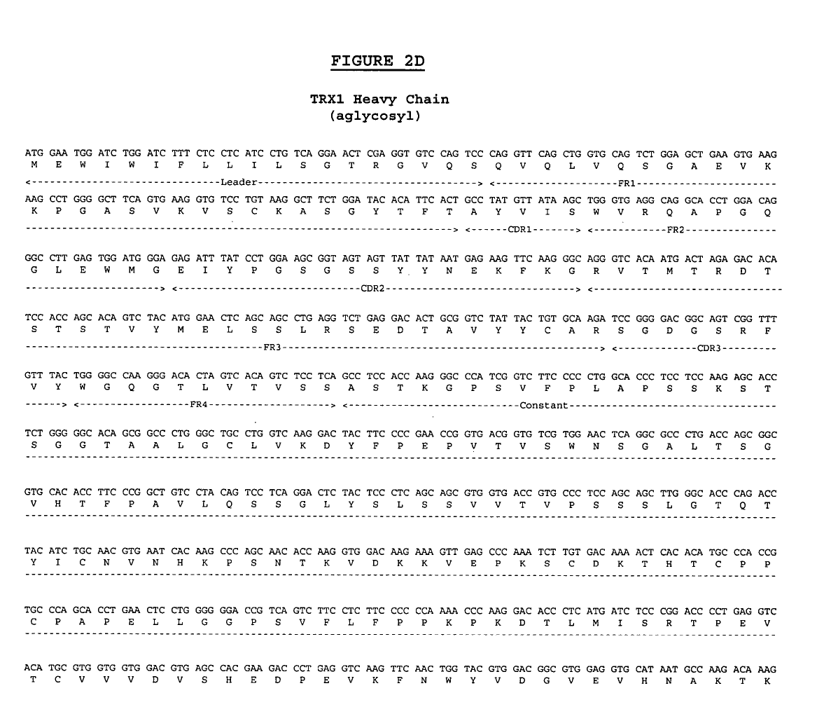

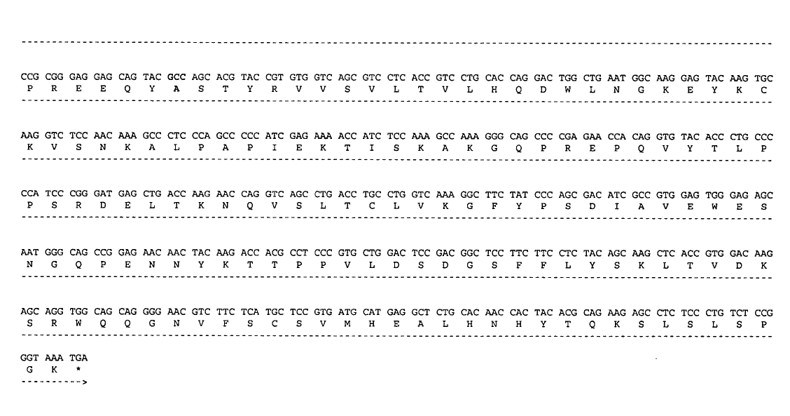

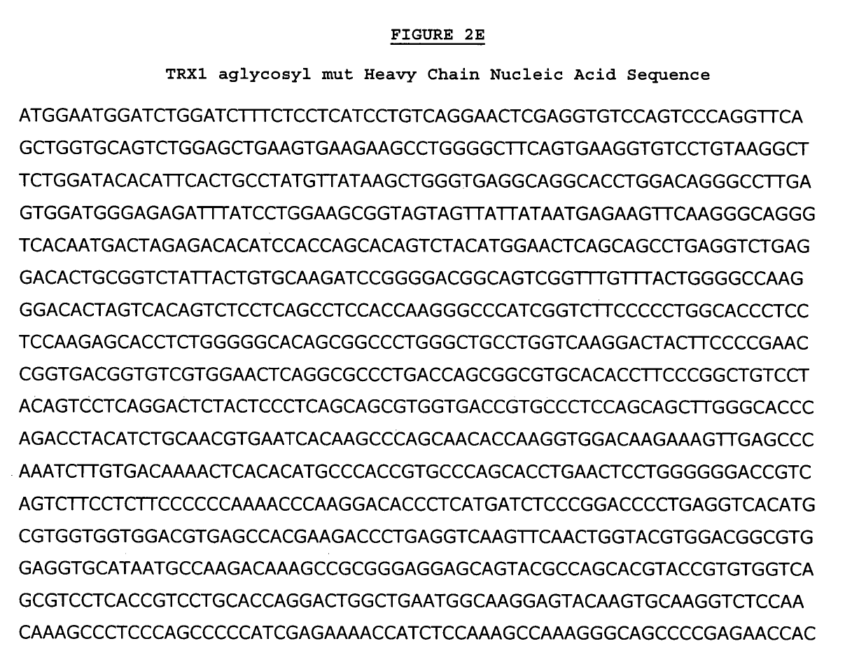

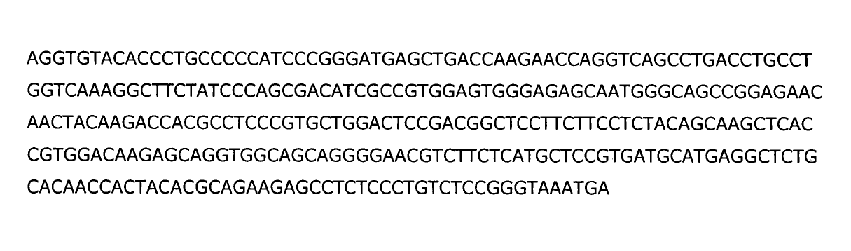

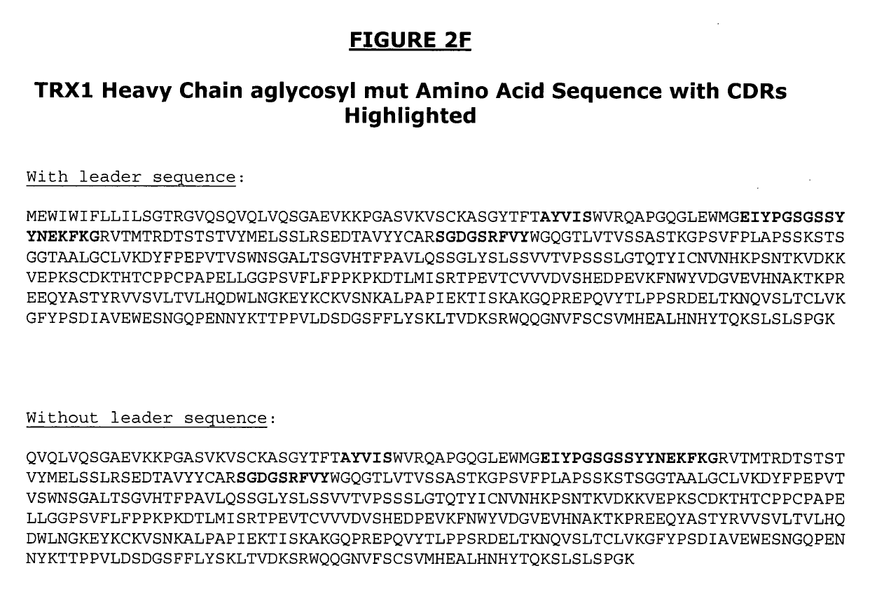

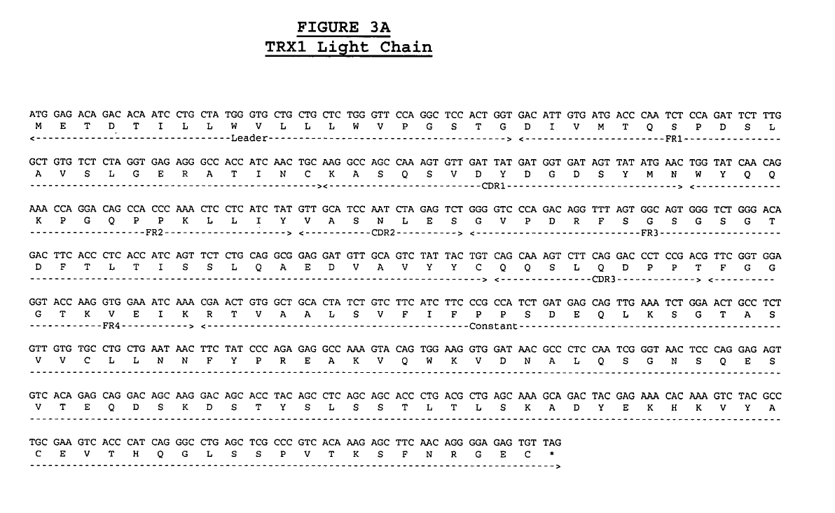

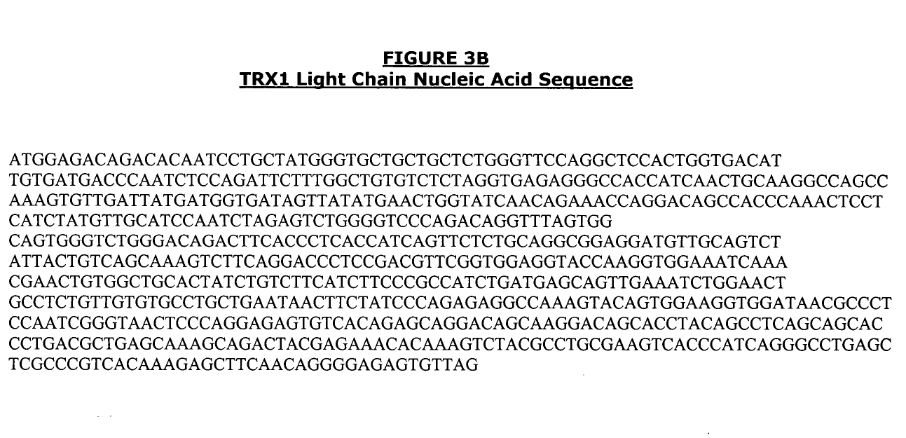

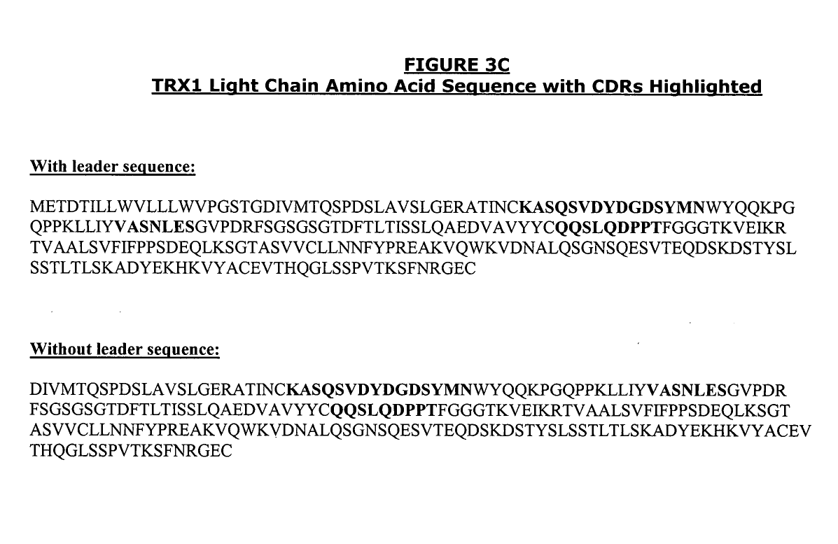

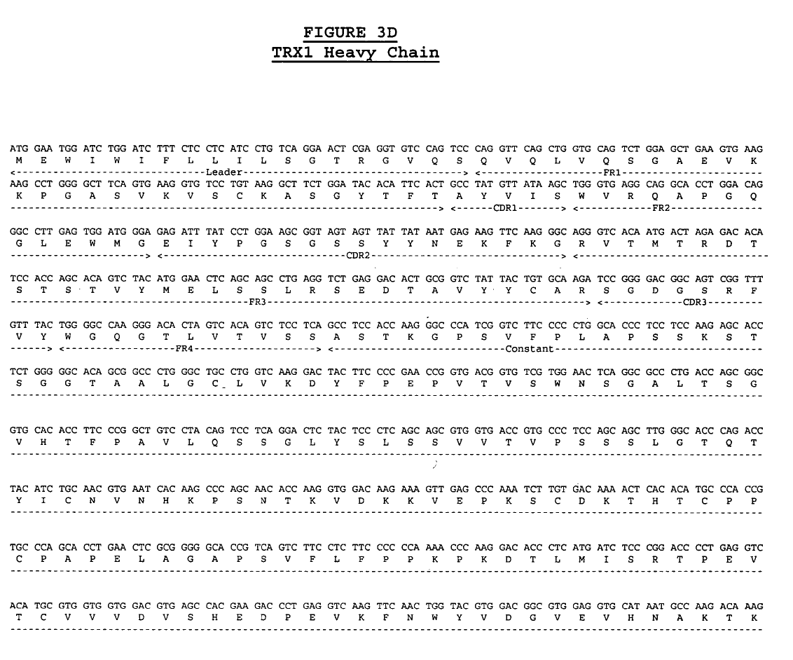

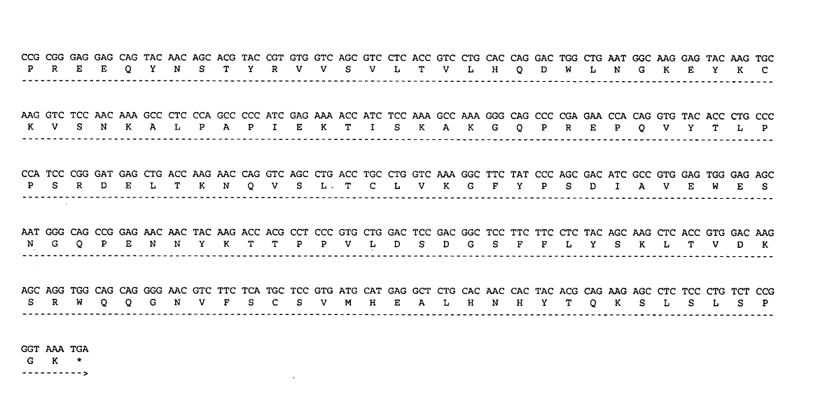

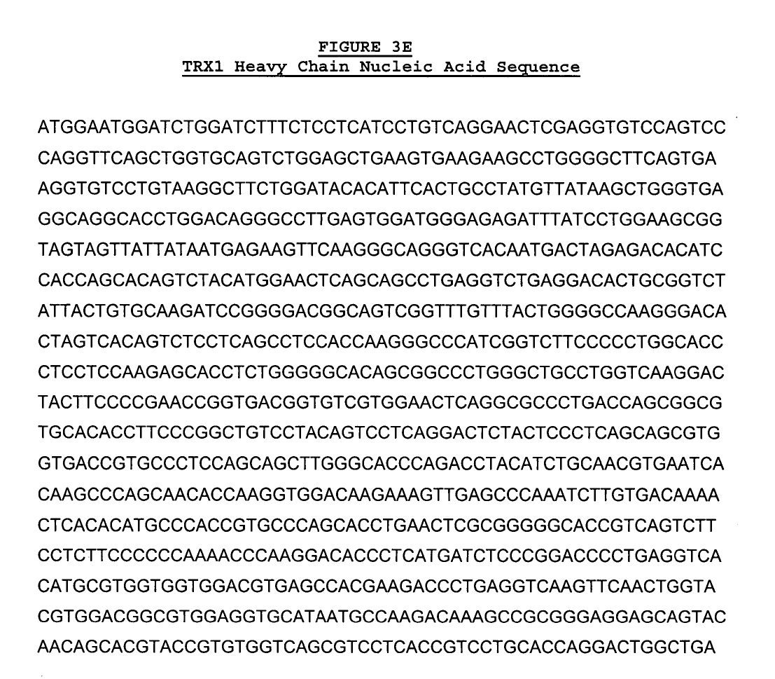

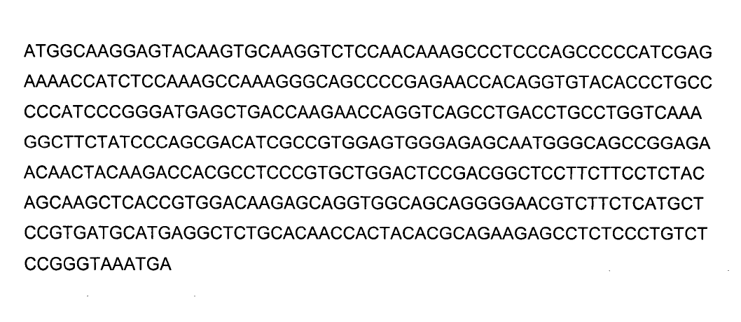

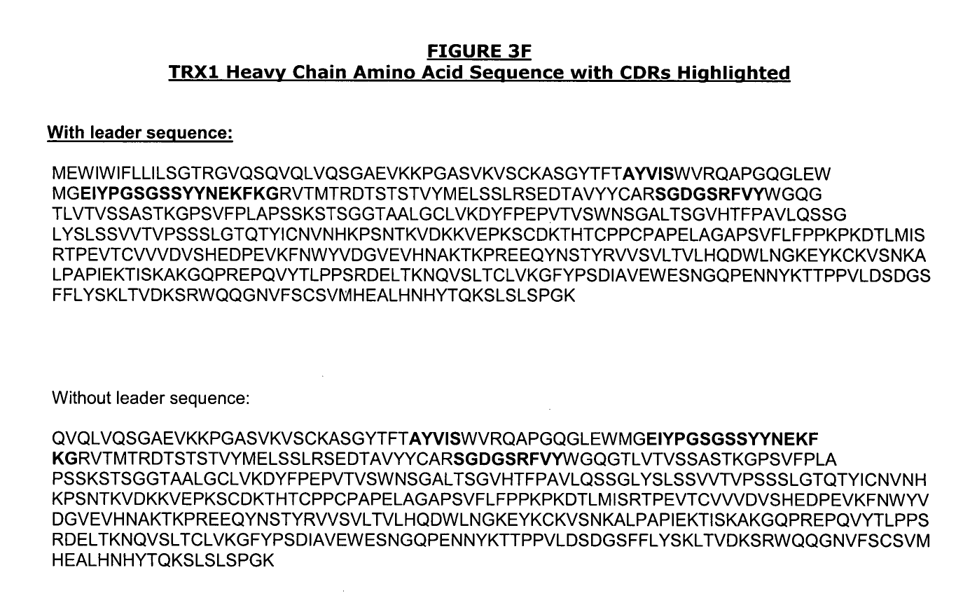

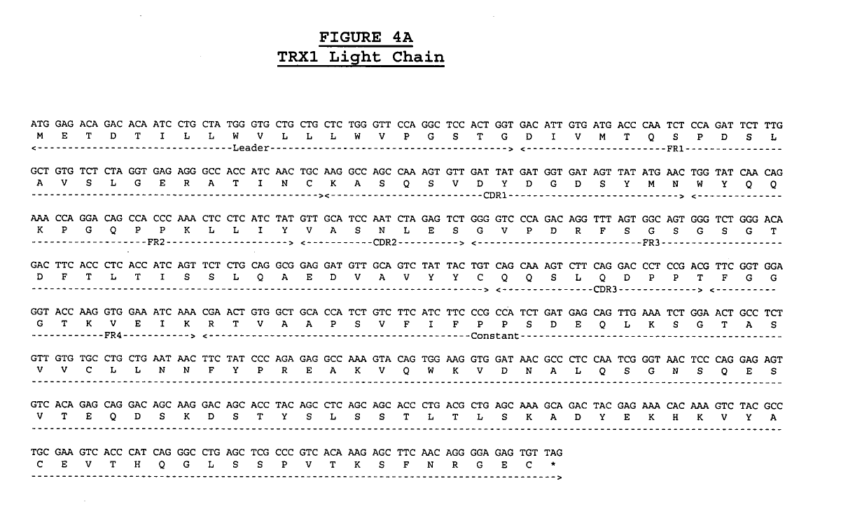

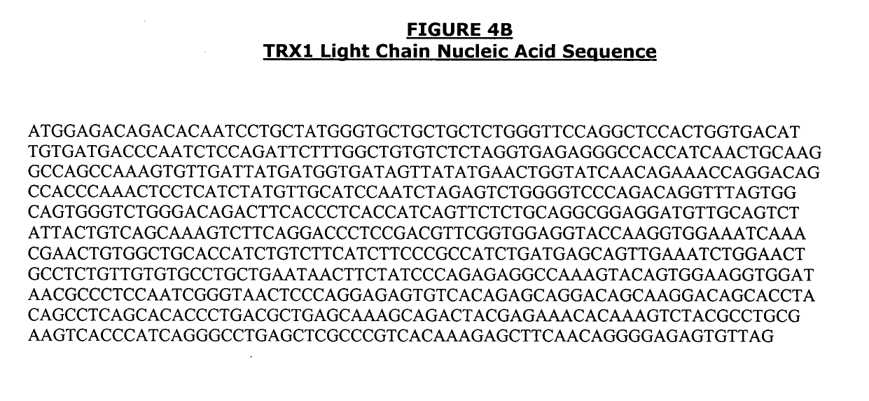

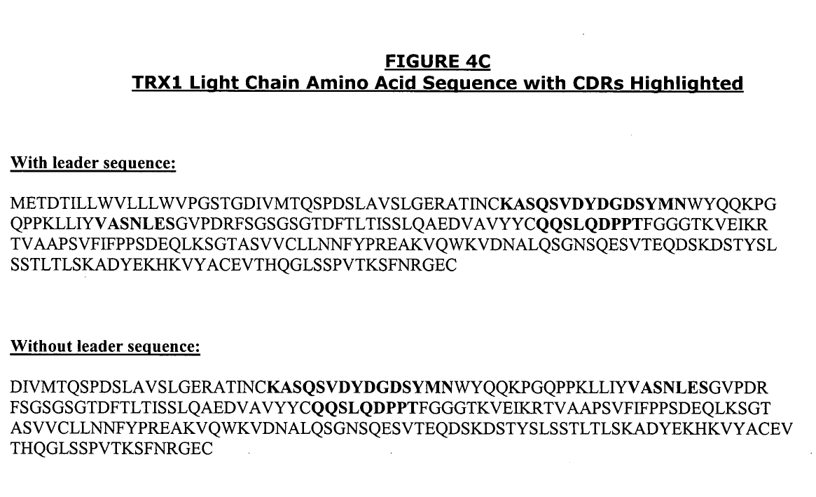

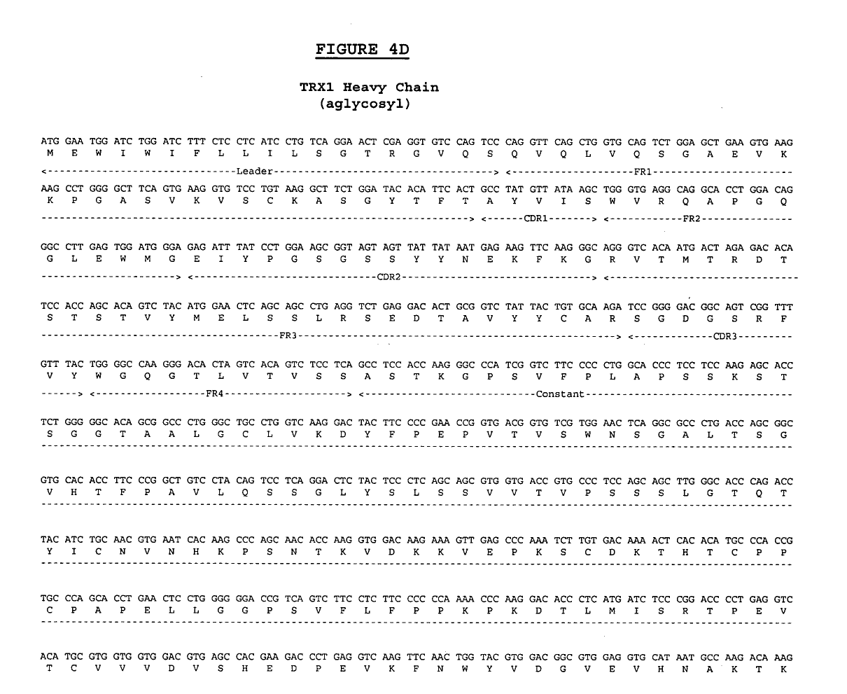

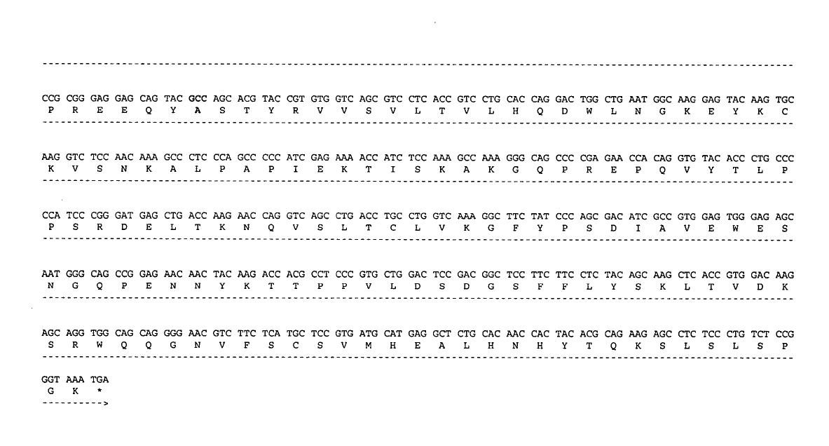

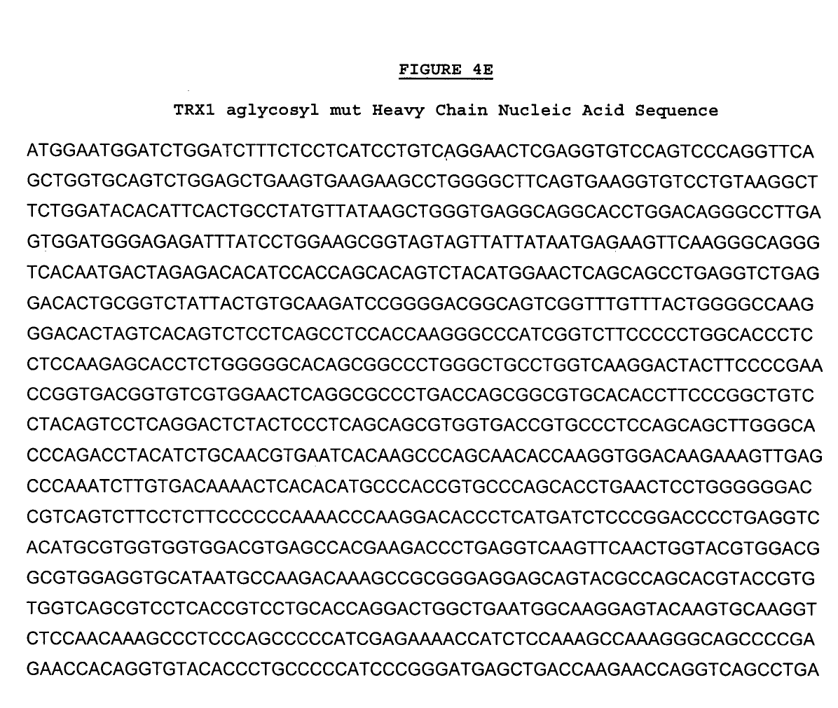

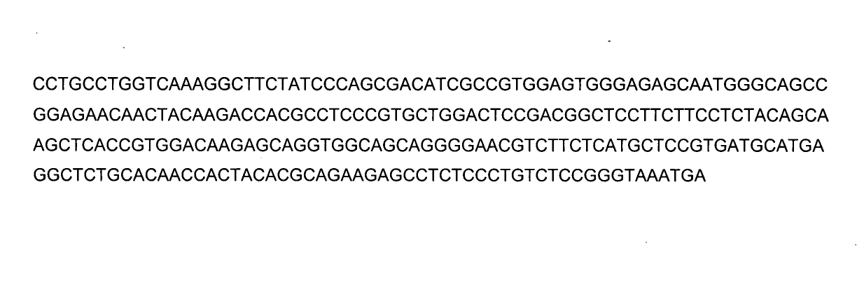

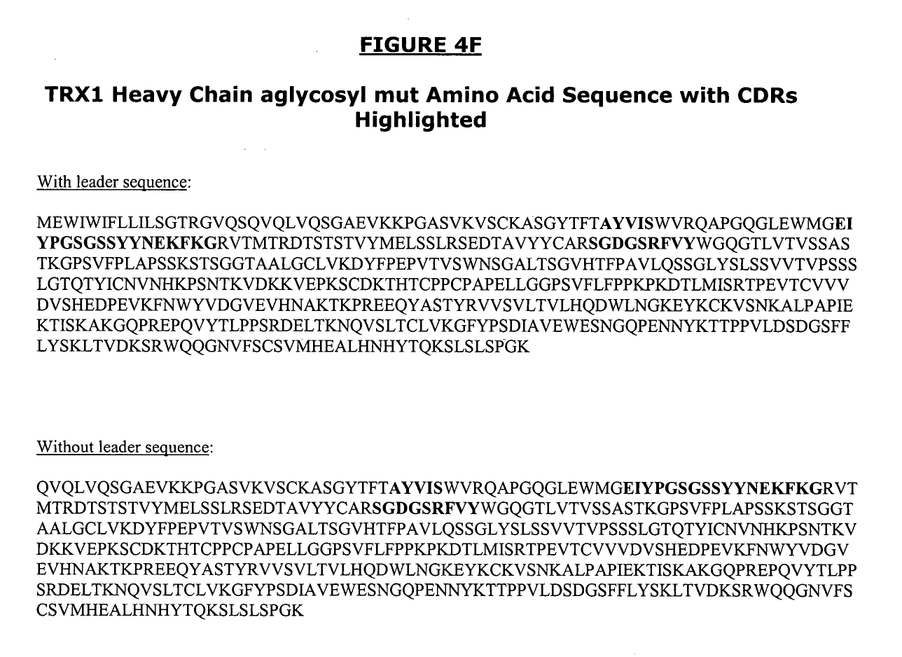

In accordance with an aspect of the present invention, such CD4 antibody (preferably a humanized antibody or fragment thereof) binds to the same epitope (or a portion thereof) on human lymphocytes as the humanized antibody selected from the group consisting of, the TRX1 humanized antibody, e.g., the components of which, e.g., light chain and heavy chain, each containing constant regions and variable regions, are depicted in FIGS. 1A-1F and correspond to SEQ ID Nos.: 1, 2, 3, 4, 5, 6, 7 and 8; the TRX1 humanized antibody, e.g., the components of which, e.g., light chain and heavy chain, each containing constant regions and variable regions, are depicted in FIGS. 2A-2F and correspond to Seq ID Nos.: 9, 10, 11, 12, 13, 14, 15, and 16; the TRX1 humanized antibody, e.g., the components of which, e.g., light chain and heavy chain, each containing constant regions and variable regions, are depicted in FIGS. 3A-3F and correspond to Seq ID Nos.: 17, 18, 19, 20, 21, 22, 23, and 24; and the TRX1 humanized antibody, e.g., the components of which, e.g., light chain and heavy chain, each containing constant regions and variable regions, are depicted in FIGS. 4A-4F and correspond to Seq ID Nos.: 25, 26,27, 28, 29, 30, 31, and 32.

The antibody is hereinafter sometimes referred to as TRX1. The term “molecule” or “antibody that binds the same epitope as TRX1” includes TRX1. The term “TRX1” includes the components of the humanized antibody, e.g., light chain and heavy chain, each containing a constant region and a variable region, e.g., amino acid sequences shown in Seq ID Nos.: 1, 3, 4, 5, 7 and 8 (FIGS. 1A, 1C, 1D and 1F), the components of the humanized antibody, e.g., light chain and heavy chain, each containing a constant region and a variable region, e.g., amino acid sequences shown in Seq ID Nos.: 9, 11, 12, 13, 15, and 16 (FIGS. 2A, 2C, 2D, and 2F), the components of the humanized antibody, e.g., light chain and heavy chain, each containing a constant region and a variable region, e.g., amino acid sequences shown in Seq ID Nos.: 17, 19, 21, 23, and 24 (FIGS. 3A, 3C, 3D and 3F), the components of the humanized antibody, e.g., light chain and heavy chain, each containing a constant region and a variable region, e.g., amino acid sequences shown in Seq ID Nos.: 25, 27, 28, 29, 31, and 32 (FIGS. 4A, 4C, 4D and 4F), and those identical thereto which may be produced, for example, by recombinant technology.

Although the preferred CD4 antibody is TRX1, from the teachings herein, one skilled in the art can produce antibodies that are equivalent to TRX1. As representative but non-limiting examples of such equivalent TRX1 antibodies there may be mentioned:

1) humanized antibodies that bind to the same epitope as TRX 1;

2) humanized antibodies that have the same CDRs as TRX1 but which have a different humanized framework and/or a different human constant region;

3) humanized antibodies that bind to the same epitope as TRX1 in which one or more amino acids of one or more of the CDRs of TRX1 have been changed (preferably but not necessarily a conservative amino acid substitution) and in which the framework may be the same framework as TRX1 or have a different humanized framework or in which one or more of the amino acids of the framework region of TRX1 have been changed and/or in which the constant region may be the same as or different from TRX1;

4) humanized antibodies that bind to the same epitope as TRX1 wherein the antibody does not bind to Fc receptors through the Fc region of the antibody.

5) humanized antibodies that bind to the same epitope as TRX1 wherein the CDRs thereof do not include a glycosylation site;

6) humanized antibodies that bind to the same epitope as TRX1 and that do not bind to Fc receptors through the Fc region of the antibody and the CDRs do not include a glycosylation site;

7) a chimeric antibody that binds to the same epitope as TRX1; and

8) a murine antibody that binds to the same epitope as TRX1.

The antibodies that are equivalent to TRX1 may be used in the same manner and for the same purposes as TRX1.

In a preferred embodiment, the CD4 antibody employed in the present invention is one which binds to the same epitope (or a part of that epitope) as the TRX1 humanized antibody. The term “binds to the same epitope as TRX1 humanized antibody” is intended to describe not only the TRX1 humanized antibody but also describes other antibodies, fragments or derivatives thereof that bind to the same such epitope as the TRX1 humanized antibody. Antibodies that bind to the same epitope as TRX1 humanized antibody can be identified using techniques known to those of ordinary skill in the art, e.g., antibody competition assays or epitope mapping.

In a preferred embodiment, the CD4 antibody does not bind to Fc receptors through the Fc region of the antibody and the CDRs do not include a glycosylation site.

The constant region may or may not include a glycosylation site. In one embodiment, the constant region includes a glycosylation site. Glycosylation signals are well known in the art. An example of a heavy chain sequence which includes a glycosylation site is shown in SEQ ID NO.:5 (FIG. 1D), SEQ ID NO.:7 (FIG. 1F) and SEQ ID NO.:8 (FIG. 1F), and SEQ ID NO.:21 (FIG. 3D), SEQ ID NO.:23 (FIG. 3F) and SEQ ID NO.:24 (FIG. 3F). In another embodiment, the constant region does not include a glycosylation site due to an asparagine (N) to an alanine (A) amino acid change. An example of a heavy chain sequence which does not include a glycosylation site is shown in SEQ ID NO.:13 (FIG. 2D), SEQ ID NO.:15(FIG. 2F) and SEQ ID NO.:16 (FIG. 2F), and SEQ ID NO.: 29 (FIG. 4D), SEQ ID NO.:31 (FIG. 4F) and SEQ ID NO.:32 (FIG. 4F).

Such other antibodies include, by way of example and not by limitation, rat, murine, porcine, bovine, human, chimeric, humanized antibodies, or fragments or derivatives thereof.

The term “fragment” as used herein means a portion of an antibody, by way of example, such portions of antibodies shall include but not be limited to CDR, Fab, or such other portions, which bind to the same epitope or any portion thereof as recognized by TRX1 .

The term “antibody” as used herein includes polyclonal and monoclonal antibodies as well as antibody fragments and derivatives, as well as antibodies prepared by recombinant techniques, such as chimeric or humanized antibodies, single chain or bispecific antibodies which bind to the same epitope or a portion thereof as recognized by the humanized antibody TRX1. The term “molecules” includes by way of example and not limitation, peptides, oligonucleotides or other such compounds derived from any source which mimic the antibody or binds to the same epitope or a portion thereof as the antibody fragment or derivative thereof.

Another embodiment of the present invention provides for a method of treating a patient who is to receive or has received a graft transplant with an effective amount of (i) at least one member selected from the group consisting of TRX1 antibody, or an antibody, or derivative or fragment thereof that bind to the same epitope (or a portion thereof) as the TRX1 antibody and (ii) a CD8 inhibiting compound. The treatment is preferably effected with the whole or intact TRX1 antibody.

In one embodiment, the antibody is TRX1 (SEQ ID Nos.:1, 2, 3, 4, 5, 6, 7, and 8; FIGS. 1A, 1B, 1C, 1D, 1E, and 1F). The TRX1 antibody, e.g., the components of the TRX1 antibody, e.g., the light chain and heavy chain, each containing variable and constant regions, which are shown in, e.g., SEQ ID Nos.: 1 (FIG. 1A), 2, (FIG. 1B), 3 (FIG. 1C, top), 4 (FIG. 1C, bottom), 5 (FIG. 1D), 6 (FIG. 1E), 7 (FIG. 1F, top), and 8 (FIG. 1F, bottom). SEQ ID No.:1 (FIG. 1A) is the amino acid sequence of the TRX1 light chain and SEQ ID No.:2 (FIG. 1B) is the nucleotide sequence of the TRX1 light chain. SEQ ID No.:3 (FIG. 1C, top) is the amino acid sequence of the TRX1 light chain, with a leader sequence. SEQ ID No.:4 (FIG. 1C, bottom) is the amino acid sequence of the TRX1 light chain, e.g., SEQ ID No.:1 or SEQ ID No.:3, without a leader sequence, e.g., amino acid residues 1-20 of SEQ ID No.:1. The TRX1 heavy chain amino acid sequence, containing a glycosylation site, e.g., amino acid residues 317-319, is shown in SEQ ID No.:5 (FIG. 1D) and the nucleotide sequence of the TRX1 heavy chain is shown in SEQ ID No.:6 (FIG. 1E). SEQ ID No.:7 (FIG. 1F, top) is the amino acid sequence of the TRX1 heavy chain with a leader sequence. SEQ ID No.:8 (FIG. 1F, bottom) is the amino acid sequence of the TRX1 heavy chain, e.g., SEQ ID No.:5 (FIG. 1D), without a leader sequence, e.g., amino acid residues 1-19 of SEQ ID No.:5 (FIG. 1D), and contains a glycosylation site, e.g., amino acid residues 298-300. TRX1 is a humanized antibody that includes modified constant regions of a human antibody, e.g., light chain amino acid residues 132-238 of SEQ ID No.:1 (FIG. 1A) or SEQ ID No.:3 (FIG. 1C, top), and amino acid residues 112-218 of SEQ ID No.:4 (FIG. 1C, bottom), and heavy chain amino acid residues 138-467 of SEQ ID No,:5 (FIG. 1D) or SEQ ID No.:7 (FIG. 1F, top) and amino acid residues 119-448 of SEQ ID No.:8 (FIG. 1F), and light and heavy chain framework and CDR regions, in which the framework regions of the light and heavy chain variable regions correspond to the framework regions of the light chain variable region, e.g., amino acid residues 21-43, 59-73, 81-112, and 122-131 of SEQ ID No.:1 (FIG. 1A) or SEQ ID No.:3 (FIG. 1C, top) and amino acid residues 1-22, 33-53, 61-92, and 102-111 of SEQ ID No.:4 (FIG. 1C), and framework regions of the heavy chain variable region, e.g., amino acid residues 20-49, 55-68, 86-117, and 127-137 of SEQ ID No.:5 or SEQ ID No.:7 (FIG. 1F, top) and amino acid residues 1-30, 36-49, 67-98, and 108-118 of SEQ ID No.:8, which are derived from a human antibody, and the CDRs of the light chain, e.g., amino acid residues 44-58. 74-80, and 113-121 of SEQ ID No.:1 or SEQ ID No.:3 (FIG. 1C, top), and amino acid residues 24-32, 54-60, and 93-101 of SEQ ID No.:4, and the CDRs of the heavy chain, e.g., amino acid residues 50-54, 69-85, and 118-126 of SEQ ID No.:5 or SEQ ID No.:7 (FIG. 1F, top) and amino acid residues 31-35, 50-66, and 99-107 of SEQ ID No.:8, which are derived from a mouse monoclonal antibody designated NSM4.7.2.4.

In another embodiment, the antibody is TRX1 (SEQ ID Nos.:17, 18, 19, 20, 21, 22, 23, and 24; FIGS. 3A, 3B, 3C, 3D, 3E, and 3F). The TRX1 antibody, e.g., the components of the TRX1 antibody, e.g., the light chain and heavy chain, each containing variable and constant regions, are shown in, e.g., SEQ ID Nos.: 17 (FIG. 3A), 18, (FIG. 3B), 19 (FIG. 3C, top), 20 (FIG. 3C, bottom), 21 (FIG. 3D), 22, (FIG. 3E) 23 (FIG. 3F, top), and 24 (FIG. 3F, bottom). SEQ ID No.:17 (FIG. 3A) is the amino acid sequence of the TRX1 light chain and SEQ ID No.:18 (FIG. 3B) is the nucleotide sequence of the TRX1 light chain. SEQ ID No.:19 (FIG. 3C, top) is the amino acid sequence of the TRX1 light chain with a leader sequence. SEQ ID No.:20 (FIG. 3C, bottom) is the amino acid sequence of the TRX1 light chain, e.g., SEQ ID No.:17, without a leader sequence, e.g., amino acid residues 1-20 of SEQ ID No.:17. The TRX1 heavy chain amino acid sequence, containing a glycosylation site, e.g., amino acid residues 317-319 of SEQ ID No.:21 (FIG. 3D) and the nucleotide sequence of the TRX1 heavy chain is shown in SEQ ID No.:22 (FIG. 3E). SEQ ID No.:23 (FIG. 3F, top) is the amino acid sequence of the TRX1 heavy chain with a leader sequence. SEQ ID No.:24 (FIG. 3F, bottom) is the amino acid sequence of the TRX1 heavy chain, e.g., SEQ ID No.:21, without a leader sequence, e.g., amino acid residues 1-19 of SEQ ID No.:21, and contains a glycosylation site, e.g., amino acid residues 298-300. TRX1 is a humanized antibody that includes modified constant regions of a human antibody, e.g., light chain amino acid residues 132-238 of SEQ ID No.:17(FIG. 3A) or SEQ ID No.:19 (FIG. 3C, top), and amino acid residues 112-218 of SEQ ID No.:20 (FIG. 3C, bottom), and heavy chain amino acid residues 138-467 of SEQ ID No.:21 (FIG. 3D) or SEQ ID No.:23 (FIG. 3F, top) and amino acid residues 119-448 of SEQ ID No.:24 (FIG. 3F, bottom), and light and heavy chain framework and CDR regions, in which the framework regions of the light and heavy chain variable regions correspond to the framework regions of the light chain variable region, e.g., amino acid residues 21-43, 59-73, 81-112, and 122-131 of SEQ ID No.:17(FIG. 3A) or SEQ ID No.:19 (FIG. 3C, top), and amino acid residues 1-22, 33-53, 61-92, and 102-111 of SEQ ID No.:20, and framework regions of the heavy chain variable region, e.g., amino acid residues 20-49, 55-68, 86-117, and 127-137 of SEQ ID No.:21(FIG. 3D) or SEQ ID No.:23 (FIG. 3F, top) and amino acid residues 1-30, 36-49, 67-98, and 108-118 of SEQ ID No.:24 (FIG. 3F, bottom), which are derived from a human antibody, and the CDRs of the light chain, e.g., amino acid residues 44-58, 74-80, and 113-121 of SEQ ID No.:17(FIG. 3A) or SEQ ID No.:19 (FIG. 3C, top), and amino acid residues 24-32, 54-60, and 93-101 of SEQ ID No.:20 (FIG. 3C, bottom), and the CDRs of the heavy chain, e.g., amino acid residues 50-54, 69-85, and 118-126 of SEQ ID No.:21 (FIG. 3D) or SEQ ID No.:23 (FIG. 3F, top) and amino acid residues 31-35, 50-66, and 99-107 of SEQ ID No.:24 (FIG. 3F, bottom), which are derived from a mouse monoclonal antibody designated NSM4.7.2.4.

In another embodiment, the antibody is TRX1 (SEQ ID Nos.:9, 10, 11, 12, 13, 14, 15, and 16; FIGS. 2A, 2B, 2C, 2D, 2E, and 2F). The TRX1 antibody, e.g., the components of the TRX1 antibody, e.g., the light chain and heavy chain, each containing variable and constant regions, are shown in, e.g., SEQ ID Nos.:9 (FIG. 2A), 10, (FIG. 2B), 11 (FIG. 2C, top), 12 (FIG. 2C, bottom), 13 (FIG. 2D), 14 (FIG. 2E), 15 (FIG. 2F, top) and 16 (FIG. 2F, bottom). SEQ ID No.:9 (FIG. 2A) is the amino acid sequence of the TRX1 light chain and SEQ ID No.:10 (FIG. 2B) is the nucleotide sequence of the TRX1 light chain. SEQ ID No.:11 (FIG. 2C) is the amino acid sequence of the TRX1 light chain with a leader sequence. SEQ ID No.:12 (FIG. 2C) is the amino acid sequence of the TRX1 light chain, e.g., SEQ ID No.:9 (FIG. 2A), without a leader sequence, e.g., amino acid residues 1-20 of SEQ ID No.:9. The TRX1 heavy chain amino acid sequence, which does not contain a glycosylation site, e.g., contains an asparagine to alanine change at amino acid residue 317, is shown in SEQ ID No.:13 (FIG. 2D) and the nucleotide sequence of the TRX1 heavy chain is shown in SEQ ID No.:14 (FIG. 2E). SEQ ID No.:15 (FIG. 2F, top) is the amino acid sequence of the TRX1 heavy chain with a leader sequence. SEQ ID No.:16 (FIG. 2F, bottom) is the amino acid sequence of the TRX1 heavy chain, e.g., SEQ ID No.:13, without a leader sequence, e.g., amino acid residues 1-19 of SEQ ID No.:13, and does not contain a glycosylation site, e.g., contains an asparagine to alanine change at amino acid residue 298. TRX1 is a humanized antibody that includes modified constant regions of a human antibody, e.g., light chain amino acid residues 132-238 of SEQ ID No.:9 (FIG. 2A) or SEQ ID No.:11 (FIG. 2C, top), and amino acid residues 112-218 of SEQ ID No.:12 (FIG. 2C, bottom), and heavy chain amino acid residues 138-467 of SEQ ID No.:13 (FIG. 2D) or SEQ ID No.:15 (FIG. 2F, top) and amino acid residues 119-448 of SEQ ID No.:16 (FIG. 2F, bottom), and light and heavy chain framework and CDR regions, in which the framework regions of the light and heavy chain variable regions correspond to the framework regions of the light chain variable region, e.g., amino acid residues 21-43, 59-73, 81-112, and 122-131 of SEQ ID No.:9(FIG. 2A) or SEQ ID No.:11 (FIG. 2C, top), and amino acid residues 1-22, 33-53, 61-92, and 102-111 of SEQ ID No.:12(FIG. 2C, bottom), and framework regions of the heavy chain variable region, e.g., amino acid residues 20-49, 55-68, 86-117, and 127-137 of SEQ ID No.:13 (FIG. 2D) or SEQ ID No.:15 (FIG. 2F, top) and amino acid residues 1-30, 36-49, 67-98, and 108-118 of SEQ ID No.:16 (FIG. 2F, bottom), which are derived from a human antibody, and the CDRs of the light chain, e.g., amino acid residues 44-58, 74-80, and 113-121 of SEQ ID No.:9 (FIG. 2A) or SEQ ID No.:11 (FIG. 2C, top), and amino acid residues 24-32, 54-60, and 93-101 of SEQ ID No.:12(FIG. 2C, bottom), and the CDRs of the heavy chain, e.g., amino acid residues 50-54, 69-85, and 118-126 of SEQ ID No.:13 (FIG. 2D) or SEQ ID No.:15 (FIG. 2F, top) and amino acid residues 31-35, 50-66, and 99-107 of SEQ ID No.:16(FIG. 2F, bottom), which are derived from a mouse monoclonal antibody designated NSM4.7.2.4.

In another embodiment, the antibody is TRX1 (SEQ ID Nos.:25, 26, 27, 28, 29, 30, 31, and 32; FIGS. 4A, 4B, 4C, 4D, 4E, and 4F). The TRX1 antibody, e.g., the components of the TRX1 antibody, e.g., the light chain and heavy chain, each containing variable and constant regions, are shown in, e.g., SEQ ID Nos.:25 (FIG. 4A), 26(FIG. 4A), 27 (FIG. 4B), 28 (FIG. 4C, top), 29 (FIG. 4C, bottom), 30 (FIG. 4A), 31 (FIG. 4A), and 32 (FIG. 4A). SEQ ID No.:25 (FIG. 4A) is the amino acid sequence of the TRX1 light chain and SEQ ID No.:26 (FIG. 4B) is the nucleotide sequence of the TRX1 light chain. SEQ ID No.:27 (FIG. 4C, top) is the amino acid sequence of the TRX1 light chain with a leader sequence. SEQ ID No.:28 (FIG. 4C, bottom) is the amino acid sequence of the TRX1 light chain, e.g., SEQ ID No.:25, without a leader sequence, e.g., amino acid residues 1-20 of SEQ ID No.:25. The TRX1 heavy chain amino acid sequence, which does not contain a glycosylation site, e.g., contains an asparagine to alanine change at amino acid residue 317, is shown in SEQ ID No.:29 (FIG. 4D) and the nucleotide sequence of the TRX1 heavy chain is shown in SEQ ID No.:30 (FIG. 4E). SEQ ID No.:31 (FIG. 4F, top) is the amino acid sequence of the TRX1 heavy chain with a leader sequence. SEQ ID No.:32 (FIG. 4F, bottom) is the amino acid sequence of the TRX1 heavy chain, e.g., SEQ ID No.:29, without a leader sequence, e.g., amino acid residues 1-19 of SEQ ID No.:29, and does not contain a glycosylation site, e.g., contains an asparagine to alanine change at amino acid residue 298. TRX1 is a humanized antibody that includes modified constant regions of a human antibody, e.g., light chain amino acid residues 132-238 of SEQ ID No.:25 (FIG. 4A) or SEQ ID No.:27 (FIG. 4C, top), and amino acid residues 112-218 of SEQ ID No.:28 (FIG. 4C, bottom), and heavy chain amino acid residues 138-467 of SEQ ID No.:29 (FIG. 4D) or SEQ ID No.:31 (FIG. 4F, top) and amino acid residues 119-448 of SEQ ID No.:32 (FIG. 4F, bottom), and light and heavy chain framework and CDR regions, in which the framework regions of the light and heavy chain variable regions correspond to the framework regions of the light chain variable region, e.g., amino acid residues 21-43, 59-73, 81-112, and 122-131 of SEQ ID No.:25 (FIG. 4A) or SEQ ID No.:27 (FIG. 4C, top), and amino acid residues 1-22, 33-53, 61-92, and 102-111 of SEQ ID No.:28 (FIG. 4C, bottom), and framework regions of the heavy chain variable region, e.g., amino acid residues 20-49, 55-68, 86-117, and 127-137 of SEQ ID No.:29 (FIG. 4D) or SEQ ID No.:31 (FIG. 4F, top) and amino acid residues 1-30, 36-49, 67-98, and 108-118 of SEQ ID No.:32 (FIG. 4F, bottom), which are derived from a human antibody, and the CDRs of the light chain, e.g., amino acid residues 44-58, 74-80, and 113-121 of SEQ ID No.:25(FIG. 4A) or SEQ ID No.:27 (FIG. 4C, top), and amino acid residues 24-32, 54-60, and 93-101 of SEQ ID No.:28 (FIG. 4C, bottom), and the CDRs of the heavy chain, e.g., amino acid residues 50-54, 69-85, and 118-126 of SEQ ID No.:29 (FIG. 4D) or SEQ ID No.:31 (FIG. 4F, top) and amino acid residues 31-35, 50-66, and 99-107 of SEQ ID No.:32 (FIG. 4F, bottom) which are derived from a mouse monoclonal antibody designated NSM4.7.2.4.

The preparation of TRX1 humanized antibody or other anti-CD4 antibody suitable for the purposes of the present invention should be apparent to those skilled in the art from the teachings herein. Such antibody may be prepared by recombinant techniques known to those skilled in the art.

This invention is further illustrated by the following examples, which should not be construed as limiting. The contents of all references, patents and published patent applications cited throughout this application, as well as the Figures, are incorporated herein by reference.

### EXAMPLES

The invention now will be described with respect to the following examples; however, the scope of the present invention is not intended to be limited thereby.

### Example 1

### Construction of TRX1 Antibody Starting from Amino Acid Sequence

A cDNA library was constructed from the mouse hybridoma NSM 4.7.2.4 using the Superscript plasmid system (Gibco/BRL, cat. no. 82485A) according to the manufacturer's suggested protocol. Heavy and light chain cDNAs were cloned from the library by DNA hybridization using as probes rat heavy and light chain gene cDNAs from the rat hybridoma YTS 177.

The rat heavy and light chain gene cDNAs of YTS 177 were isolated from the expression vector pHA Pr-1 as BamH1/Sal 1 fragments and labeled with ³²p and used independently to screen the NSM 4.7.2.4. cDNA library using standard molecular biology techniques (Sambrook, et al., Molecular Cloning, A. Laboratory Manual, 3rd edition, Cold Spring Harbor Laboratory Press, Cold Spring Harbor, N.Y. (2001); Ausubel, et al., Current Protocols in Molecular Biology, John Wiley & Sons, New York (2001).) Sequence analysis of the cDNAs derived from the NSM 4.7.2.4 cDNA library confirmed the NSM 4.7.2.4 heavy chain to be mouse gamma-1 subclass and the NSM 4.7.2.4 light chain to be kappa. The NSM 4.7.2.4 heavy and light V regions (VH and VL, respectively) were reshaped to the human VH and VL regions with the “best fit” or highest sequence similarity in the framework regions to that of the mouse. For the light chain, human antibody HSIGKAW (from EMBL) with a sequence similarity of 79% was used (LA Spatz et al., 1990 J. Immunol. 144:2821-8). The sequence of HSIGKAW VL (SEQ ID No.35) is:

MVLQTQVFISLLLWISGAYGDIVMTQSPDSLAVSLGERATINCKSS
QSLLYSSNNKNYLAWYQQKPGQPPKLLIYWASTRESGVPDRFSG
SGSGTDFTLTISSLQAEDVAVYYCQQYYSTPPMFGQGTKVEIKRT

D start of framework 1

Q changed to G

For the heavy chain, human antibody A32483 (From GenBank) with a sequence similarity of 74% was used (Larrick, et al., Biochem. Biophys. Res. Comm., Vol. 160, pgs. 1250-1256 (1989)). The sequence of A32483 VH (SEQ ID No.36) is:

LLAVAPGAHSQVQLVQSGAEVKKPGASVKVSCKASGYTFTNYY
MHWVRQAPGQGLEWMGIINPSGNSTNYAQKFQGRVTMTRDTST
STVYMELSSLRSEDTAVYYCAREKLATTIFGVLI
ITGMDYWGQGTLVTVSSGSASA

Q start of framework 1

For the humanization process, anti-CD4 light chain clone 77.53.1.2 (insert size 1 kb) and anti-CD4 heavy chain clone 58.59.1 (insert size 1.7 kb) were chosen from the cDNA library and inserts isolated from the pSport vector as Sal I/Not I fragments and cloned into M13 mp18 vector to produce single stranded DNA for sequencing and template for mutagenesis. The humanization of NSM 4.7.2.4 was performed by site-directed mutagenesis of the mouse cDNA using a kit from Amersham International (RPN 1523) according to the manufacturer's suggested protocol.

Mutagenesis of the VL gene framework regions was performed using five oligonucleotides ranging in length from 29 to 76 bases. The oligos used were:

Primer #1998 (SEQ ID No.37) 76 bases
5′-TGA CAT TGT GAT GAC CCA ATC TCC AGA TTC TTT GGC
TGT GTC TCT AGG TGA GAG GGC CAC CAT CAA CTG CAA
GGC C
Primer #1999(SEQ ID No.38) 29 bases
5′-TGA ACT GGT ATC AAC AGA AAC CAG GAC AG
Primer #2000(SEQ ID No.39) 28 bases
5′-AGA GTC TGG GGT CCC AGA CAG GTT TAG T
Primer #2001 (SEQ ID No.40) 42 bases
5′-GTC TTC AGG ACC CTC CGA CGT TCG GTG GAG GTA CCA
AGC TGG
Primer #2008 (SEQ ID No.41) 52 bases
5′-CAC CCT CAC CAT CAG TTC TCT GCA GGC GGA GGA TGT
TGC AGT CTA TTA GTG T

The oligos were phosphorylated and mutagenesis performed in three steps using no more than two oligos per step to introduce changes according to the following procedure:

(1) Annealing phosphorylated mutant oligos to ssDNA template

(2) Polymerization

(3) Filtration to remove single-stranded DNA

(4) Nicking non mutant strand with Nci I

(5) Digestion of non-mutant strand with Exo III

(6) Repolymerization of gapped DNA

(7) Transformation of competent JM101

(8) Sequencing of clones

Mutations were confirmed by single strand DNA sequencing using M13 primers −20 and −40 and also the mutagenic primers # 1999 and # 2000.

A Sal I site at the 5′ end of the variable region was changed to Hind III by linker oligos #2334 and #2335 to allow cloning of the variable region as a Hind III/Kpn I fragment into the light chain constant region of CAMPATH-1H.

Primer #2334 (SEQ ID No.42) 24 bases
5′-AGC TTT ACA GTT ACT GAG CAC ACA
Primer #2335 (SEQ ID No.43) 24 bases
5′-TCG ATG TGT GCT CAG TAA CTG TAA

Mutagenesis of the VH gene framework regions was performed using five oligonucleotides ranging in length from 24 to 75 bases. The oligos used were:

Primer #2003 (SEQ ID No.44) 75 bases
5′-GGT TCA GCT GGT GCA GTC TGG AGC TGA AGT GAA GAA
GCC TGG GGC TTC AGT GAA GGT GTC CTG TAA GGC TTC TGG
Primer #2004 (SEQ ID No.45) 52 bases
5′-AGC TGG GTG AGG CAG GCA CCT GGA CAG GGC CTT GAG
TGG ATG GGA GAG ATT T
Primer #2005 (SEQ ID No.46) 60 bases
5′-CAA GGG CAG GGT CAC AAT GAC TAG AGA CAC ATC CAC
CAG CAC AGT CTA CAT GGA ACT CAG
Primer #2006(SEQ ID No.47) 44 bases
5′CAG CCT GAG GTC TGA GGA CAC TGC GGT CTA TTA CTG
TGC AAG A
Primer #2007 (SEQ ID No.48) 24 bases
5′-GCC AAG GGA CAC TAG TCA CTG TGT

Mutagenesis was carried out as described above for the light chain again using no more than two oligos at a time to introduce the changes. Mutations were confirmed by single strand DNA sequencing using M13 primers −20 and −40 as well as the mutagenic primers #2002 and #2004.

Primer #2002 was used to correct a reading frame error in starting clone 58.59.1.

Primer #2002 (SEQ ID No.49) 39 bases

5′-ACT CTA ACC ATG GAA TGG ATC TGG ATC TTT CTC CTC ATC

Primer #2380 was used to correct extra mutation added by #2004 which was missed in the first sequencing.

Primer #2380 (SEQ ID No.50) 39 bases
5′-TCA CTG CCT ATG TTA TAA GCT GGG TGA GGC AGG CAC CTG

As with the light chain, the heavy chain 5′ Sal I site was changed to Hind III using linker oligo's #2334 and #2335 to allow cloning of the heavy chain variable region as Hind III/Spe I (site introduced by primer #2007) fragment into the heavy chain constant region of CAMPATH-1H.

Construction of Heavy Chain

The following samples of DNA were used:

1. Plasmid 1990. Human gamma-1 heavy chain constant region gene cloned into pUC18 (obtained from Martin Sims, Wellcome Foundation Ltd).

2. Plasmid 2387: Reshaped heavy chain of NSM 4.7.2.4 containing human framework regions and mouse gamma 1 constant region.

A Sal I site in the reshaped CD4 heavy chain was altered to a Hind III site. The variable region gene was excised by digestion with Hind III/Spe I and ligated with the constant region gene in plasmid 1990 to give a complete humanized heavy chain (plasmid 2486). The heavy chain gene was cut out of this plasmid with Hind III/EcoR I and ligated with the expression vector pEE6.

Construction of Light Chain

The following samples of DNA were used.

1. Plasmid 2028; CAMPATH-1H light chain gene cloned into M13 mp18 at Sal I/BamH I restriction site.

2. Plasmid 2197; Reshaped light chain of NSM 4.7.2.4 containing human framework regions and mouse kappa constant region. A Kpn I site already had been introduced between variable and constant portions of this gene.

A Kpn I restriction site was introduced into the CAMPATH 1 H light chain gene corresponding to the site in plasmid 2197 and an EcoR I site was introduced at the 3′ end of the constant region. The constant region gene was excised from this plasmid (2502) by digestion with Hind III/Kpn I.

Meanwhile a Sal I site in plasmid 2197 was changed to a Hind III site (this step had to be repeated because a frame-shift mutation inadvertently was introduced the first time). The new plasmid (2736) was digested with Hind III/Kpn I. The CD4 variable region fragment was cloned into a plasmid containing the kappa constant region gene from plasmid 2502 to give a complete humanized light chain (plasmid 2548). The light chain gene was cut out from this plasmid with Hind III/EcoR I and ligated with the expression vector pEE12 to give plasmid 2798.

Ligation of Heavy and Light Chains and Expression in NSO Cells

The heavy chain gene was excised from the pEE6 vector by digestion with Sal I/Bgl II and cloned into the light chain pEE12 vector which had been digested with BamH I/Sal I.

The final vector construct was checked by restriction digests with Hind III, EcoR I, Sal I, BamH I, BgI II and Spe I for the presence of the expected fragments, including 700 bp light chain, 1400 bp heavy chain, 2300 bp fragment of pEE6 and 7000 bp fragment of pEE 12.

The pEE12 vector was linearized by digestion with Sal I and transferred into NSO cells by electroporation, following a standard protocol (Celltech 1991) except that the selection medium was slightly modified, being based on IMDM rather than DMEM. Transfectants were selected in medium lacking glutamine, supplemented with dialysed FCS, ribonucleosides, glutamic acid, and asparagine as recommended.

The transfection mixes were cultured in three 96-well plates, and of 36 growing wells which were tested, 5 were strongly positive for production of human heavy and light chains (18 others were positive for one or other, or weakly positive for both).

A clone, designated SDG/B7B.A.7 was selected and stored frozen but no further characterization has been done on this wild type antibody.

Construction of Mutant IgG1 Antibody Designated to Abolish Effector Functions

Due to concerns about side effects of other CD4 antibodies reported in various clinical trials, it was considered desirable to avoid the possibility of engaging Fc receptors. Human IgG4 is thought to have minimal Fc binding or complement-activating ability. However, experiments have show that it does engage Fc receptors in some individuals (Greenwood et al., Eur. J. Immunol., Vol. 23, pgs. 1098-1104, 1993), and clinical studies with a human IgG4 variant to CAMPATH-1H have demonstrated an ability to kill cells in vivo (Isaacs et al., Clin. Exp. Immunol., Vol. 106, pgs. 427-433 (1996)). To eliminate the possibility of binding Fc receptors, constructs were made with mutations in the IgG1 heavy chain constant region.

TRX 1 has the mutations Leu²³⁶ to Ala and Gly²³⁸ to Ala, as shown in SEQ ID Nos.:5 and 6, and SEQ ID Nos.:21 and 22. These particular residues were chosen because they were predicted to disrupt maximally binding to all three types of human Fc receptors for IgG. Either mutation is sufficient to reduce binding to Fc(RI (Woof, et al., Mol. Immunol, Vol. 332, pgs. 563-564, 1986; Duncan, et al., Nature, Vol. 332, pgs. 563-564 1988; Lund, et al., J. Immunol, Vol. 147, pgs. 2657-2662 1991) or Fc(RII (Lund et al., 1991; Sarmay et al., Mol. Immunol., Vol. 29, pgs. 633-639 1992) whereas Gly²³⁸ to Ala has the biggest effect on binding to Fc(RIII (Sarmay et al., 1992).

The following samples of DNA were used.

1. Plasmid 2555 and Plasmid 2555 Mut.; the humanized V_{H} region of NSM 4.7.2.4 cloned into pEE6 expression vector at a Hind III/Spe I restriction site. Plasmid 2555 then was mutated by site directed mutagenesis such that amino acid residue Asn¹⁰¹ was changed to Asp¹⁰¹, as shown in SEQ ID Nos.:5 and 6, and SEQ ID Nos.:21 and 22. The resulting plasmid is plasmid 2555 Mut.

2. Plasmid 2798; the humanized V_{H} region of NSM 4.7.2.4 joined to human kappa constant regions to give approx 700 bp fragment cloned into pEE12 expression vector at a Hind III/EcoR I.

3. Plasmid MF4260; the human IgG1 heavy chain associated with the humanized CD18 V_{H} region, having the mutations Leu²³⁶ to Ala and Gly²³⁸ to Ala as well as a Spe I restriction site introduced into framework region 4, cloned into pUC18.

The purpose of the Spe I restriction site is to allow separation and recombination of different variable regions.

The CD18 V_{H} region gene was excised from plasmid MF 4260 by digestion with Spe I and Hind III and the remaining vector, now having only the relevant heavy chain constant region, was purified using Geneclean. It was ligated with the humanized V_{H} region DNA of NSM 4.7.2.4 which had been isolated from plasmid 2555 Mut in the same way. The product was used to transform “Sure” cells and colonies were checked for the presence of the expected 1400 bp complete heavy chain insert.

The complete V_{H} and constant region insert was excised from the pUC vector by digestion with Hind III and EcoR I. The 1400 bp fragment was purified using QiaexII (Qiagen) and then ligated in turn into the vector pEE6, which had previously been cut with the same enzymes.

The next step was to excise the CD4 heavy chain genes from the pEE6 vector and clone them into pEE12, already containing the humanized CD4 light chain gene (plasmid 2798). The pEE6 vector was digested with Sal I and BgI II and the pEE12 vector was digested with Sal I and BamH I to create the appropriate sites for re-ligation.

The final vector construct was checked by restriction digests with Hind III, EcoR I, Sal I and Spe I for the presence of the expected fragment, i.e., 700 bp light chain, 1400 bp heavy chain, 2300 bp fragment of pEE6, and 7000 bp fragment of pEE12.

The pEE12 vector was linearized by digestion with Sal I and transfected into NSO cells by electroporation as above. The transfection mixes were cultured in six 96-well plates, and of 90 growing wells which were tested, all were positive for production of human heavy and light chains. At this stage a sample of the pEE12 vector DNA was digested with Sal I, precipitated with ethanol and transferred to the Therapeutic Antibody Centre (TAC).

Target Cells for Final Transfection

NSO cells were obtained directly from the ECACC (clone CB1782, accession number 85110503). A master cell bank (MCB) was prepared at the Therapeutic Antibody Centre, Churchill Hospital, Oxford, England.

Transfection and Selection of Final Transfectant

The pEE12 vector was transfected into NSO cells from the MCB by electroporation as hereinabove described. A total of 2×10⁷ cells were transfected with 80 μg of linearized plasmid DNA in a final volume of 2.0 ml. The transfection mix was plated out in twelve 96-well plates and fed with selective medium according to the standard protocol (The Cell Tech Glutamine Synthetase Gene Expression System, Version 2 — Expression from Myeloma Cells, Revision 6.) Six plates received selective medium containing 10(M methionine sulfoximine (MSX).

Purification of the Antibody

Culture supernatant is purified by using a Biopilot chromatography system (Pharmacia) in three steps as follows:

(1) Affinity chromatography on a column of Protein A-Sepharose Fast Flow

(2) Ion exchange chromatography on S-Sepharose Fast Flow

(3) Size exclusion chromatography on Superdex 20.

The purified product was filtered and pooled into a single biocontainer.

Throughout the purification process, precautions are taken to ensure that the system remains aseptic. All buffers and reagents are passed through a 0.2 micron membrane filter and the purified product is also passed through a 0.2 micron filter before being pooled. After a batch of antibody has been processed, the entire chromatography system and columns are sanitized with 0.5M NaOH, washed with sterile PBS and stored in 20% ethanol. Before it is used again, the ethanol is washed out with sterile PBS and a complete trial run is carried out. Samples of buffers and column eluates are checked for endotoxin level.

### Example 2

### Construction of TRX1 Antibody Starting from Nucleotide Sequence

Cloning of Human Constant Regions

Heavy Chain Constant Region

The human gamma 1 heavy chain constant region (IgG1) is amplified from human leukocyte cDNA (QUICK-Clone™ cDNA Cat. No. 7182-1, Clontech) using the following primer set and cloned into pCR-Script (Stratagene). The plasmid containing the human gamma 1 heavy chain constant region in pCR-Script is designated pHCγ-1.

primer hcγ-1 (SEQ ID No.51)

| | |
|---|---|
| | Spe I |
| 5′ primer: | 5′-ACT AGT CAC AGT CTC CTC AGC |

primer hcγ-2 (SEQ ID No.52)

| | |
|---|---|
| | EcoR I |
| 3′ primer: | 5′-GAA TTC ATT TAC CCG GAG ACA G |

Non-Fc binding mutations (Leu²³⁶ Ala, Gly²³⁸ Ala) are made in the heavy chain constant region by site-directed mutagenesis using the following primer and the Transformer™ Site-Directed Mutagenesis Kit from Clontech (Cat. No. K1600-1). The plasmid containing the human gamma 1 heavy chain non-Fc binding mutant constant region in pCR-Script is designated pHCγ-1Fcmut.

| | | |
|---|---|---|
| | primer hcγ-3(SEQ ID No.53) | |
| | Fc mut oligo: | 5′-CCG TGC CCA GCA CCT GAA CTC GCG |
| GGG | | GCA CCG TCA GTC TTC CTC CCC C |

Light Chain Constant Region

The human kappa light chain constant region is amplified from human leukocyte cDNA (QUICK-Clone™ cDNA Cat. No. 7182-1, Clontech) using the following primer set and cloned into pCR-Script (Stratagene). The plasmid containing the human kappa light chain constant region in pCR-Script is designated pLCκ-1.

primer lcκ-1 (SEQ ID No.54)

| | |
|---|---|
| | Kpn I |
| 5′ primer: | 5′-GGT ACC AAG GTG GAA ATC AAA CGA AC |

primer lcκ-2 (SEQ ID No.55)

| | |
|---|---|
| | Hind III |
| 3′ primer: | 5′-AAG CTT CTA ACA CTC TCC CCT GTT G |

Synthesis, Construction and Cloning of TRX1 Variable Regions

The heavy and light chain variable regions are constructed from a set of partially overlapping and complementary synthetic oligonucleotides encompassing the entire variable regions. The oligonucleotide set used for each variable region is shown below.

Heavy Chain Variable Region Synthetic Oligonucleotides

Coding Strand Heavy Chain Variable Region Primers

| | |
|---|---|
| | primer hv-1 (1-72) + 6 nucleotide linker (SEQ ID No.56) |
| | 5′-aagctt ATG GAA TGG ATC TGG ATC TTT CTC CTC ATC CTG |
| TCA | GGA ACT CGA GGT GTC CAG TCC CAG GTT CAG CTG GTG |
| | primer hv-2 (120-193) (SEQ ID No.57) |
| | 5′-C TGT AAG GCT TCT GGA TAC ACA TTC ACT GCC TAT GTT |
| ATA | AGC TGG GTG AGG CAG GCA CCT GGA CAG GGC CTT G |
| | primer hv-3 (223-292) (SEQ ID No.58) |
| | 5′-GGT AGT AGT TAT TAT AAT GAG AAG TTC AAG GGC AGG |
| GTC | ACA ATG ACT AGA GAC ACA TCC ACC AGC ACA G |
| | primer hv-4 (322-399) (SEQ ID No.59) |
| | 5′-GAG GAC ACT GCG GTC TAT TAC TGT GCA AGA TCC GGG |
| GAC | GGC AGT CGG TTT GTT TAC TGG GGC CAA GGG ACA CTA GT |
| | primer hv-5 (140-51) (SEQ ID No.60) |
| | 5′-GTG TAT CCA GAA GCC TTA CAG GAC ACC TTC ACT GAA |
| GCC | CCA GGC TTC TTC ACT TCA GCT CCA GAC TGC ACC AGC TGA |
| ACC | TGG GAC TGG |
| | primer hv-6 (246-170) (SEQ ID No.61) |
| | 5′-CTT CTC ATT ATA ATA ACT ACT ACC GCT TCC AGG ATA |
| AAT CTC | |
| | TCC CAT CCA CTC AAG GCC CTG TCC AGG TGC CTG CC |
| | primer hv-7 (342-272) (SEQ ID No.62) |
| | 5′-GTA ATA GAC CGC AGT GTC CTC AGA CCT CAG GCT GCT |
| GAG | TTC CAT GTA GAC TGT GCT GGT GGA TGT GTC TC |

Non-Coding Strand Heavy Chain Variable Region Primers

Light Chain Variable Region Synthetic Oligonucleotides

Coding Strand Light Chain Variable Region Primers

| | |
|---|---|
| | primer lv-1 (1-63) + 6 nucleotide linker (SEQ ID No.63) |
| | 5′-gaattc ATG GAG ACA GAC ACA ATC CTG CTA TGG GTG CTG |
| CTG | CTC TGG GTT CCA GGC TCC ACT GGT GAC |
| | primer lv-2 (93-158) (SEQ ID No.64) |
| | 5′-GGC TGT GTC TCT AGG TGA GAG GGC CAC CAT CAA CTG |
| CAA | GGC CAG CCA AAG TGT TGA TTA TGA TGG |
| | primer lv-3 (184-248) (SEQ ID No.65) |
| | 5′-CAG AAA CCA GGA CAG CCA CCC AAA CTC CTC ATC TAT |
| GTT | GCA TCC AAT CTA GAG TCT GGG GTG CC |
| | primer lv-4 (275-340) (SEQ ID No.66) |
| | 5′-GGA CAG ACT TCA CCC TCA CCA TCA GTT CTC TGC AGG |
| CGG | AGG ATG TTG CAG TCT ATT ACT GTC AGC |
| | primer lv-5 (109-43) (SEQ ID No.67) |
| | 5′-CAC CTA GAG ACA CAG CCA AAG AAT CTG GAG ATT GGG |
| TCA | TCA CAA TGT CAC CAG TGG AGC CTG GAA C |
| | primer lv-6 (203-138) (SEQ ID No.68) |
| | 5′-GGT GGC TGT CCT GGT TTC TGT TGA TAC CAG TTC ATA |
| TAA CTA | |
| | TCA CCA TCA TAA TCA ACA CTT TGG |
| | primer lv-7 (294-228) (SEQ ID No.69) |
| | 5′-GGT GAG GGT GAA GTG TGT CCC AGA CCC ACT GCC ACT |
| AAA | CCT GTC TGG GAC CCC AGA CTC TAG ATT G |
| | primer lv-8 (378-319) (SEQ ID No.70) |
| | 5′-GGT ACC TCC ACC GAA CGT CGG AGG GTC CTG AAG ACT |
| TTG | CTG ACA GTA ATA GAC TGC AAC |

Non-Coding Strand Light Chain Variable Region Primers

After HPLC purification and removal of organic solvents the oligonucleotides are resuspended in TE pH8.0 and phosphorylated. An aliquot of each oligonucleotide in the respective variable region set then are combined in equal molar amounts. The oligonucleotide mixtures are heated to 68° C. for 10 minutes and allowed to cool slowly to room temperature. The annealed oligonucleotides then are extended to produce double stranded variable region DNA fragments. For the extension, dNTPs are added to a final concentration of 0.25 mM followed by an appropriate volume of 5×T4 DNA polymerase buffer [165 mM Tris acetate, pH 7.9, 330 mM sodium acetate, 50 mM magnesium acetate, 500 (g/ml BSA, 2.5 mM DTT] and 4 units of T4 DNA polymerase. The mixture is incubated at 37° C. for 1 hour followed by heat inactivation of the T4 DNA polymerase at 65° C. for 5 minutes.

The double stranded DNA is ethanol precipitated and resuspended in the same volume of TE pH 8.0. An appropriate volume of 5×T4 DNA ligase buffer [250mM Tris-HCl, pH7.6, 50 mM MgCl₂, 5 mM ATP, 5 mM DTT, 25% w/v polyethylene glycol-8000] then is added to the double stranded DNA followed by 2 units of T4 DNA ligase and the mixture incubated for 1 hour at 37° C. to ligate the extended fragments. The T4 DNA ligase then is heat inactivated at 65° C. for 10 minutes. The variable region DNA fragments then are phenol extracted, ethanol precipitated, and resuspended in TE, pH 8.0 and cloned into pCR-Script (Stratagene). The resulting plasmid containing the heavy chain variable region is designated pHV-1 and the plasmid containing the light chain variable region was designated pLV-1.

The final heavy and light chain expression vectors are constructed in pcDNA 3.1 (Invitrogen). For the heavy chain expression vector, the Fc mutated constant region is released from plasmid pHC-1Fcmut by digestion with Spe I and EcoR I and isolated by agarose gel electrophoresis. The heavy chain variable region is released from plasmid pHV-1 by digestion with Hind III and Spe I and isolated by agarose gel electrophoresis. The two fragments in equal molar amounts are ligated into the Hind III/EcoR I sites of pcDNA3.1 (+) (Invitrogen) using standard molecular biology techniques. The resulting TRX1 heavy chain expression vector is designated pTRX1/HC.

Similarly, for the light chain expression vector, the light chain constant region is released from plasmid pLC-1 by digestion with Kpn I and Hind III followed by agarose gel purification. The light chain variable region is released from pLV-1 by digestion with EcoR I and Kpn I followed by agarose gel purification. The two light chain fragments in equal molar amounts are ligated into the EcoR I/Hind III sites of pcDNA3.1(−) (Invitrogen) using standard molecular biology techniques yielding the TRX1 light chain expression vector pTRX1/LC.

For production of TRX1 antibody, the TRX1 heavy chain and TRX1 light chain expression plasmids are cotransfected into CHO cells using standard molecular biology techniques.

### Example 3

### Construction of Aglycosylated TRX1 Antibody

A humanized antibody, e.g., the components of the humanized antibody, e.g., light chain and heavy chain, each containing constant regions and variable regions, e.g., amino acid sequences are shown in Seq ID Nos.: 9, 11, 12, 13, 15, and 16 (FIGS. 2A, 2C, 2D, and 2F), and is produced by a procedure similar to that of Example 1. The humanized antibody is an aglycosylated antibody.

### Example 4

### Construction of Aglycosylated TRX1 Antibody

A humanized antibody, e.g., the components of the humanized antibody, e.g., light chain and heavy chain, each containing constant regions and variable regions, e.g., amino acid sequences are shown in Seq ID Nos.: 25, 27, 28, 29, 31, and 32 (FIGS. 4A, 4C, 4D, and 4F), and is produced by a procedure similar to that of Example 1. The humanized antibody is an aglycosylated antibody.

### Example 5

### Treatment of a Primate with TRX1 Antibody

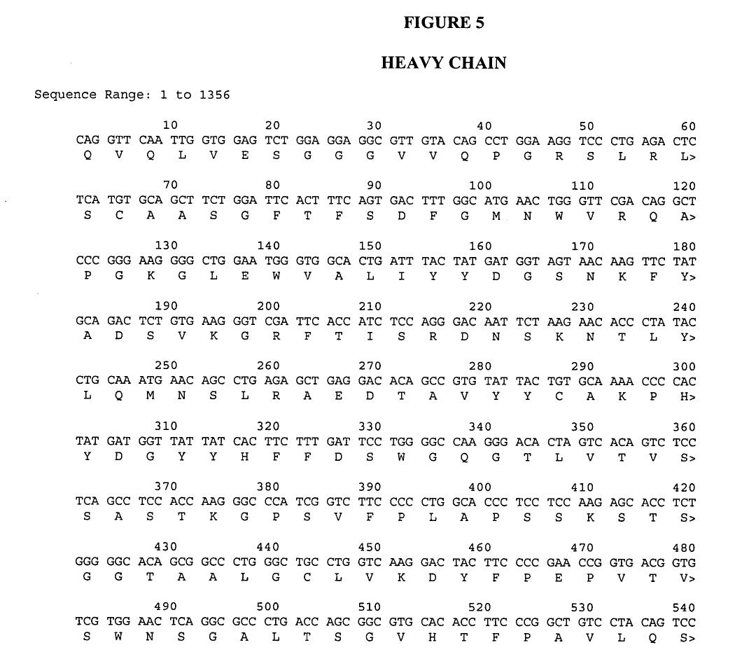

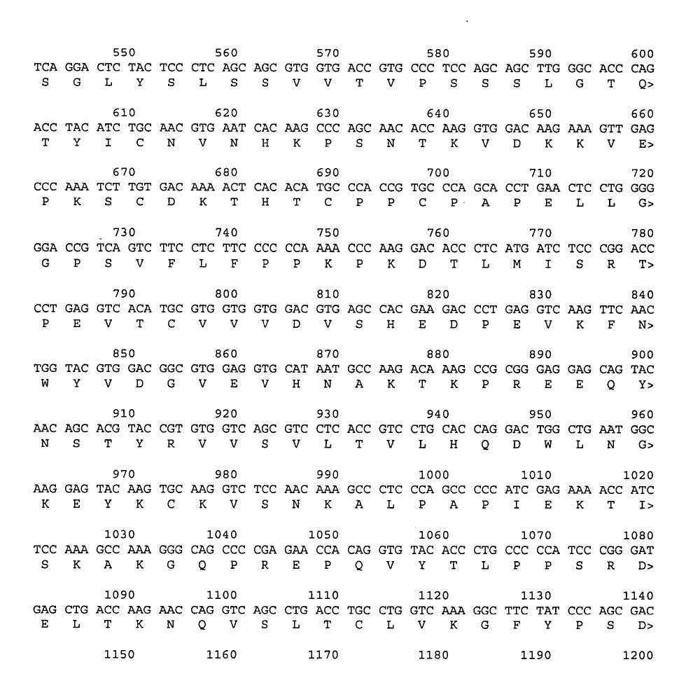

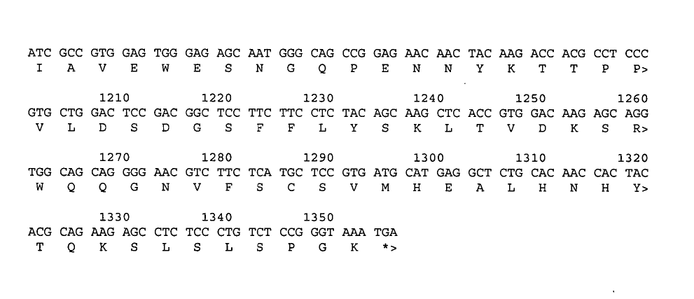

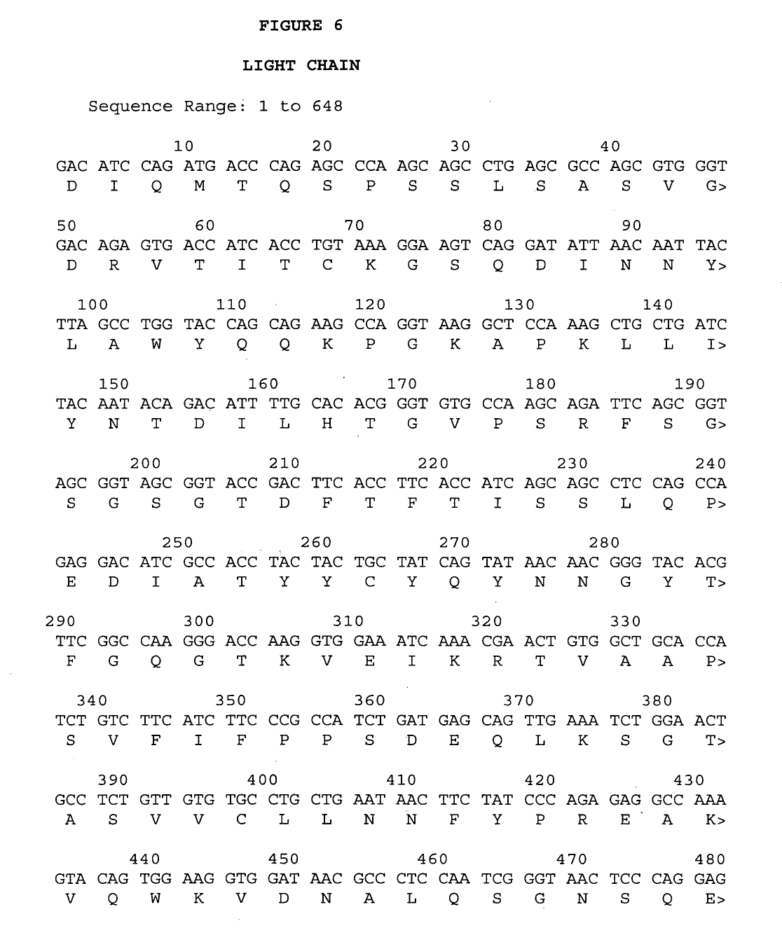

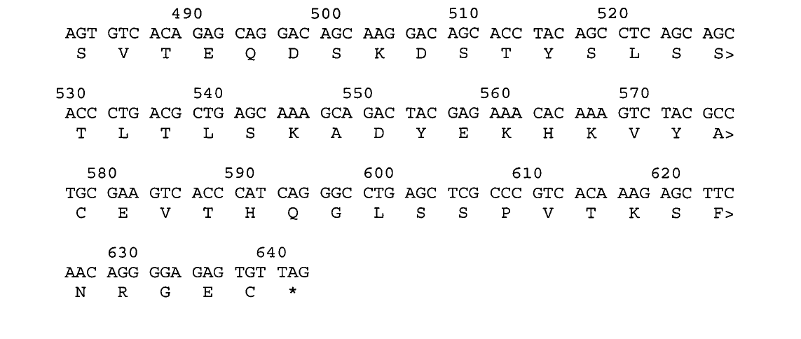

A baboon having a weight of 4.6 kg received a mismatched kidney transplant from another baboon on day 1 and was treated with both the CD4 antibody, e.g., the humanized antibody, e.g., the components of the humanized antibody, e.g., light chain and heavy chain, each containing a constant region and a variable region, e.g., amino acid sequences shown in Seq ID Nos.: 9, 11, 12, 13, 15, and 16, and with a depleting humanized CD8 antibody, the amino acid sequences of which is shown in SEQ ID Nos.:33 (FIG. 5) and 34 (FIG. 6) in accordance with the following Protocol of Table 1.

The animal has survived for more than 80 days without receiving an immunosuppressant. In addition except for a period of about two days, creatinine levels were below 2 mg/dL.

Numerous modifications and variations of the invention are possible in light of the above teachings; therefore, within the scope of the appended claims, the invention may be practiced otherwise than as particularly described.

TABLE 1
Protocol
Study 2

| | DAYS | | | | | | | | | | | | | | | |
|---|---|---|---|---|---|---|---|---|---|---|---|---|---|---|---|---|
| ACTION | 0 | 1 | 2 | 3 | 4 | 5 | 6 | 7 | 8 | 9 | 10 | 11 | 12 | 13 | 14 | 15 |
| Treatments | | | | | | | | | | | | | | | | |
| Renal transplantation | | X | | | | | | | | | | | | | | |
| CD4 antibody (iv)² | X | | X | | X | | | X | | X | | X | | | | |
| CD8 antibody (iv)³ | X | | X | | X | | | X | | X | | X | | | | |

²CD4 antibody 40 mg/kg on day 0 and 20 mg/kg for all other doses was given by iv infusion over 1 hour
³CD8 antibody 6 mg/kg given as an iv bolus after the CD4 antibody infusion

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

What is claimed is:

1. A process for treating a primate to induce tolerance to at least one antigen comprising:
administering to the primate at least one anti-CD4 antibody or CD4 binding fragment thereof and at least one compound that inhibits CD8+ T cells each in an amount and for a time effective to induce tolerance against at least one antigen, said anti-CD4 antibody or fragment being present in said primate when said antigen is present in said primate and said anti-CD4 antibody being administered in an initial dose of at least 40 mg/kg, such that tolerance against said antigen is induced.

2. The process of claim 1 wherein the antigen is a foreign antigen.

3. A process for inducing tolerance in a primate to a transplanted antigen, comprising:
administering to a primate at least one anti-CD4 antibody or CD4 binding fragment thereof and at least one compound that inhibits CD8+ T cells each in an amount and for a time effective to induce tolerance against the transplant, said anti-CD4 antibody or fragment being present in said primate when said transplanted antigen is present in said primate and said anti-CD4 antibody being administered in an initial dose of at least 40 mg/kg, such that tolerance to the transplanted antigen is induced.


SEQUENCE LISTING (70 sequences)

SEQ ID NO: 1 - DNA, 717 bp, Artificial Sequence
  Chimeric Sequence
atggagacagacacaatcctgctatgggtgctgctgctctgggttccaggctccactggtgacattgtgatgacccaatctccagattctttggctgtgtctctaggtgagagggccaccatcaactgcaaggccagccaaagtgttgattatgatggtgatagttatatgaactggtatcaacagaaaccaggacagccacccaaactcctcatctatgttgcatccaatctagagtctggggtcccagacaggtttagtggcagtgggtctgggacagacttcaccctcaccatcagttctctgcaggcggaggatgttgcagtctattactgtcagcaaagtcttcaggaccctccgacgttcggtggaggtaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - DNA, 716 bp, Artificial Sequence
  Chimeric Sequence
atggagacagacacaatcctgctatgggtgctgctgctctgggttccaggctccactggtgacattgtgatgacccaatctccagattctttggctgtgtctctaggtgagagggccaccatcaactgcaaggccagccaaagtgttgattatgatggtgatagttatatgaactggtatcaacagaaaccaggacagccacccaaactcctcatctatgttgcatccaatctagagtctggggtcccagacaggtttagtggcagtgggtctgggacagacttcaccctcaccatcagttctctgcaggcggaggatgttgcagtctattactgtcagcaaagtcttcaggaccctccgacgttcggtggaggtaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcacaccctgacgctgagcaaagcagactacgagaaacacaaagtctacgcctgcgaagtcacccatcagggcctgagctcgcccgtcacaaagagcttcaacaggggagagtgttag

SEQ ID NO: 3 - protein, 238 aa, Artificial Sequence
  Chimeric Sequence
METDTILLWVLLLWVPGSTGDIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 218 aa, Artificial Sequence
  Chimeric Sequence
DIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1404 bp, Artificial Sequence
  Chimeric Sequence
atggaatggatctggatctttctcctcatcctgtcaggaactcgaggtgtccagtcccaggttcagctggtgcagtctggagctgaagtgaagaagcctggggcttcagtgaaggtgtcctgtaaggcttctggatacacattcactgcctatgttataagctgggtgaggcaggcacctggacagggccttgagtggatgggagagatttatcctggaagcggtagtagttattataatgagaagttcaagggcagggtcacaatgactagagacacatccaccagcacagtctacatggaactcagcagcctgaggtctgaggacactgcggtctattactgtgcaagatccggggacggcagtcggtttgtttactggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - DNA, 1404 bp, Artificial Sequence; identical to SEQ ID NO: 5
  Chimeric Sequence

SEQ ID NO: 7 - protein, 467 aa, Artificial Sequence
  Chimeric Sequence
MEWIWIFLLILSGTRGVQSQVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 448 aa, Artificial Sequence
  Chimeric Sequence
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 717 bp, Artificial Sequence
  Chimeric Sequence
atggagacagacacaatcctgctatgggtgctgctgctctgggttccaggctccactggtgacattgtgatgacccaatctccagattctttggctgtgtctctaggtgagagggccaccatcaactgcaaggccagccaaagtgttgattatgatggtgatagttatatgaactggtatcaacagaaaccaggacagccacccaaactcctcatctatgttgcatccaatctagagtctggggtcccagacaggtttagtggcagtgggtctgggacagacttcaccctcaccatcagttctctgcaggcggaggatgttgcagtctattactgtcagcaaagtcttcaggaccctccgacgttcggtggaggtaccaaggtggaaatcaaacgaactgtggctgcactatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 10 - DNA, 717 bp, Artificial Sequence; identical to SEQ ID NO: 9
  Chimeric Sequence

SEQ ID NO: 11 - protein, 238 aa, Artificial Sequence
  Chimeric Sequence
METDTILLWVLLLWVPGSTGDIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAALSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 218 aa, Artificial Sequence
  Chimeric Sequence
DIVMTQSPDSLAVSLGERATINCKASQSVDYDGDSYMNWYQQKPGQPPKLLIYVASNLESGVPDRFSGSGSGTDFTLTISSLQAEDVAVYYCQQSLQDPPTFGGGTKVEIKRTVAALSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1404 bp, Artificial Sequence
  Chimeric Sequence
atggaatggatctggatctttctcctcatcctgtcaggaactcgaggtgtccagtcccaggttcagctggtgcagtctggagctgaagtgaagaagcctggggcttcagtgaaggtgtcctgtaaggcttctggatacacattcactgcctatgttataagctgggtgaggcaggcacctggacagggccttgagtggatgggagagatttatcctggaagcggtagtagttattataatgagaagttcaagggcagggtcacaatgactagagacacatccaccagcacagtctacatggaactcagcagcctgaggtctgaggacactgcggtctattactgtgcaagatccggggacggcagtcggtttgtttactggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - DNA, 1404 bp, Artificial Sequence; identical to SEQ ID NO: 13
  Chimeric Sequence

SEQ ID NO: 15 - protein, 467 aa, Artificial Sequence
  Chimeric Sequence
MEWIWIFLLILSGTRGVQSQVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 448 aa, Artificial Sequence
  Chimeric Sequence
QVQLVQSGAEVKKPGASVKVSCKASGYTFTAYVISWVRQAPGQGLEWMGEIYPGSGSSYYNEKFKGRVTMTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 717 bp, Artificial Sequence; identical to SEQ ID NO: 9
  Chimeric Sequence

SEQ ID NO: 18 - DNA, 717 bp, Artificial Sequence; identical to SEQ ID NO: 9
  Chimeric Sequence

SEQ ID NO: 19 - protein, 238 aa, Artificial Sequence; identical to SEQ ID NO: 11
  Chimeric Sequence

SEQ ID NO: 20 - protein, 218 aa, Artificial Sequence; identical to SEQ ID NO: 12
  Chimeric Sequence

SEQ ID NO: 21 - DNA, 1404 bp, Artificial Sequence; identical to SEQ ID NO: 5
  Chimeric Sequence

SEQ ID NO: 22 - DNA, 1404 bp, Artificial Sequence; identical to SEQ ID NO: 5
  Chimeric Sequence

SEQ ID NO: 23 - protein, 467 aa, Artificial Sequence; identical to SEQ ID NO: 7
  Chimeric Sequence

SEQ ID NO: 24 - protein, 448 aa, Artificial Sequence; identical to SEQ ID NO: 8
  Chimeric Sequence

SEQ ID NO: 25 - DNA, 717 bp, Artificial Sequence; identical to SEQ ID NO: 1
  Chimeric Sequence

SEQ ID NO: 26 - DNA, 716 bp, Artificial Sequence; identical to SEQ ID NO: 2
  Chimeric Sequence

SEQ ID NO: 27 - protein, 238 aa, Artificial Sequence; identical to SEQ ID NO: 3
  Chimeric Sequence

SEQ ID NO: 28 - protein, 218 aa, Artificial Sequence; identical to SEQ ID NO: 4
  Chimeric Sequence

SEQ ID NO: 29 - DNA, 1404 bp, Artificial Sequence; identical to SEQ ID NO: 13
  Chimeric Sequence

SEQ ID NO: 30 - DNA, 1404 bp, Artificial Sequence; identical to SEQ ID NO: 13
  Chimeric Sequence

SEQ ID NO: 31 - protein, 467 aa, Artificial Sequence; identical to SEQ ID NO: 15
  Chimeric Sequence

SEQ ID NO: 32 - protein, 448 aa, Artificial Sequence; identical to SEQ ID NO: 16
  Chimeric Sequence

SEQ ID NO: 33 - DNA, 1356 bp, Artificial Sequence
  Chimeric Sequence
caggttcaattggtggagtctggaggaggcgttgtacagcctggaaggtccctgagactctcatgtgcagcttctggattcactttcagtgactttggcatgaactgggttcgacaggctcccgggaaggggctggaatgggtggcactgatttactatgatggtagtaacaagttctatgcagactctgtgaagggtcgattcaccatctccagggacaattctaagaacaccctatacctgcaaatgaacagcctgagagctgaggacacagccgtgtattactgtgcaaaaccccactatgatggttattatcacttctttgattcctggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - DNA, 642 bp, Artificial Sequence
  Chimeric Sequence
gacatccagatgacccagagcccaagcagcctgagcgccagcgtgggtgacagagtgaccatcacctgtaaaggaagtcaggatattaacaattacttagcctggtaccagcagaagccaggtaaggctccaaagctgctgatctacaatacagacattttgcacacgggtgtgccaagcagattcagcggtagcggtagcggtaccgacttcaccttcaccatcagcagcctccagccagaggacatcgccacctactactgctatcagtataacaacgggtacacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 35 - protein, 135 aa, Artificial Sequence
  Synthetic Oligonucleotide
MVLQTQVFISLLLWISGAYGDIVMTQSPDSLAVSLGERATINCKSSQSLLYSSNNKNYLAWYQQKPGQPPKLLIYWASTRESGVPDRFSGSGSGTDFTLTISSLQAEDVAVYYCQQYYSTPPMFGQGTKVEIKRT

SEQ ID NO: 36 - protein, 142 aa, Artificial Sequence
  Synthetic Oligonucleotide
LLAVAPGAHSQVQLVQSGAEVKKPGASVKVSCKASGYTFTNYYMHWVRQAPGQGLEWMGIINPSGNSTNYAQKFQGRVTMTRDTSTSTVYMELSSLRSEDTAVYYCAREKLATTIFGVLIITGMDYWGQGTLVTVSSGSASA

SEQ ID NO: 37 - DNA, 76 bp, Artificial Sequence
  Synthetic Oligonucleotide
tgacattgtgatgacccaatctccagattctttggctgtgtctctaggtgagagggccaccatcaactgcaaggcc

SEQ ID NO: 38 - DNA, 29 bp, Artificial Sequence
  Synthetic Oligonucleotide
tgaactggtatcaacagaaaccaggacag

SEQ ID NO: 39 - DNA, 28 bp, Artificial Sequence
  Synthetic Oligonucleotide
agagtctggggtcccagacaggtttagt

SEQ ID NO: 40 - DNA, 42 bp, Artificial Sequence
  Synthetic Oligonucleotide
gtcttcaggaccctccgacgttcggtggaggtaccaagctgg

SEQ ID NO: 41 - DNA, 52 bp, Artificial Sequence
  Synthetic Oligonucleotide
caccctcaccatcagttctctgcaggcggaggatgttgcagtctattagtgt

SEQ ID NO: 42 - DNA, 24 bp, Artificial Sequence
  Synthetic Oligonucleotide
agctttacagttactgagcacaca

SEQ ID NO: 43 - DNA, 24 bp, Artificial Sequence
  Synthetic Oligonucleotide
tcgatgtgtgctcagtaactgtaa

SEQ ID NO: 44 - DNA, 75 bp, Artificial Sequence
  Synthetic Oligonucleotide
ggttcagctggtgcagtctggagctgaagtgaagaagcctggggcttcagtgaaggtgtcctgtaaggcttctgg

SEQ ID NO: 45 - DNA, 52 bp, Artificial Sequence
  Synthetic Oligonucleotide
agctgggtgaggcaggcacctggacagggccttgagtggatgggagagattt

SEQ ID NO: 46 - DNA, 60 bp, Artificial Sequence
  Synthetic Oligonucleotide
caagggcagggtcacaatgactagagacacatccaccagcacagtctacatggaactcag

SEQ ID NO: 47 - DNA, 43 bp, Artificial Sequence
  Synthetic Oligonucleotide
cagcctgaggtctgaggacactgcggtctattactgtgcaaga

SEQ ID NO: 48 - DNA, 24 bp, Artificial Sequence
  Synthetic Oligonucleotide
gccaagggacactagtcactgtgt

SEQ ID NO: 49 - DNA, 39 bp, Artificial Sequence
  Synthetic Oligonucleotide
actctaaccatggaatggatctggatctttctcctcatc

SEQ ID NO: 50 - DNA, 39 bp, Artificial Sequence
  Synthetic Oligonucleotide
tcactgcctatgttataagctgggtgaggcaggcacctg

SEQ ID NO: 51 - DNA, 21 bp, Artificial Sequence
  Synthetic Oligonucleotide
actagtcacagtctcctcagc

SEQ ID NO: 52 - DNA, 22 bp, Artificial Sequence
  Synthetic Oligonucleotide
gaattcatttacccggagacag

SEQ ID NO: 53 - DNA, 49 bp, Artificial Sequence
  Synthetic Oligonucleotide
ccgtgcccagcacctgaactcgcgggggcaccgtcagtcttcctccccc

SEQ ID NO: 54 - DNA, 26 bp, Artificial Sequence
  Synthetic Oligonucleotide
ggtaccaaggtggaaatcaaacgaac

SEQ ID NO: 55 - DNA, 25 bp, Artificial Sequence
  Synthetic Oligonucleotide
aagcttctaacactctcccctgttg

SEQ ID NO: 56 - DNA, 78 bp, Artificial Sequence
  Synthetic Oligonucleotide
aagcttatggaatggatctggatctttctcctcatcctgtcaggaactcgaggtgtccagtcccaggttcagctggtg

SEQ ID NO: 57 - DNA, 74 bp, Artificial Sequence
  Synthetic Oligonucleotide
ctgtaaggcttctggatacacattcactgcctatgttataagctgggtgaggcaggcacctggacagggccttg

SEQ ID NO: 58 - DNA, 70 bp, Artificial Sequence
  Synthetic Oligonucleotide
ggtagtagttattataatgagaagttcaagggcagggtcacaatgactagagacacatccaccagcacag

SEQ ID NO: 59 - DNA, 77 bp, Artificial Sequence
  Synthetic Oligonucleotide
gaggacactgcggtctattactgtgcaagatccggggacggcagtcggtttgtttactggggccaagggacactagt

SEQ ID NO: 60 - DNA, 90 bp, Artificial Sequence
  Synthetic Oligonucleotide
gtgtatccagaagccttacaggacaccttcactgaagccccaggcttcttcacttcagctccagactgcaccagctgaacctgggactgg

SEQ ID NO: 61 - DNA, 77 bp, Artificial Sequence
  Synthetic Oligonucleotide
cttctcattataataactactaccgcttccaggataaatctctcccatccactcaaggccctgtccaggtgcctgcc

SEQ ID NO: 62 - DNA, 71 bp, Artificial Sequence
  Synthetic Oligonucleotide
gtaatagaccgcagtgtcctcagacctcaggctgctgagttccatgtagactgtgctggtggatgtgtctc

SEQ ID NO: 63 - DNA, 69 bp, Artificial Sequence
  Synthetic Oligonucleotide
gaattcatggagacagacacaatcctgctatgggtgctgctgctctgggttccaggctccactggtgac

SEQ ID NO: 64 - DNA, 66 bp, Artificial Sequence
  Synthetic Oligonucleotide
ggctgtgtctctaggtgagagggccaccatcaactgcaaggccagccaaagtgttgattatgatgg

SEQ ID NO: 65 - DNA, 65 bp, Artificial Sequence
  Synthetic Oligonucleotide
cagaaaccaggacagccacccaaactcctcatctatgttgcatccaatctagagtctggggtccc

SEQ ID NO: 66 - DNA, 66 bp, Artificial Sequence
  Synthetic Oligonucleotide
ggacagacttcaccctcaccatcagttctctgcaggcggaggatgttgcagtctattactgtcagc

SEQ ID NO: 67 - DNA, 67 bp, Artificial Sequence
  Synthetic Oligonucleotide
cacctagagacacagccaaagaatctggagattgggtcatcacaatgtcaccagtggagcctggaac

SEQ ID NO: 68 - DNA, 66 bp, Artificial Sequence
  Synthetic Oligonucleotide
ggtggctgtcctggtttctgttgataccagttcatataactatcaccatcataatcaacactttgg

SEQ ID NO: 69 - DNA, 67 bp, Artificial Sequence
  Synthetic Oligonucleotide
ggtgagggtgaagtctgtcccagacccactgccactaaacctgtctgggaccccagactctagattg

SEQ ID NO: 70 - DNA, 60 bp, Artificial Sequence
  Synthetic Oligonucleotide
ggtacctccaccgaacgtcggagggtcctgaagactttgctgacagtaatagactgcaac